(19) United States
(12) Patent Application Publication
(10) Pub. No.: US 2026/0325640 A1
(43) Pub. Date: Oct. 22, 2026

(54) METHODS FOR INHIBITING GALECTIN-DEPENDENT IMMUNOSUPPRESSION OF ADOPTIVE T CELL THERAPY

(71) Applicants: THE FLORIDA INTERNATIONAL UNIVERSITY BOARD OF TRUSTEES, MIAMI, FL (US); THE TRUSTEES OF THE UNIVERSITY OF PENNSYLVANIA, PHILADELPHIA, PA (US)

(72) Inventors: CHARLES J. DIMITROFF, CORAL GABLES, FL (US); AVERY D. POSEY, PHILADELPHIA, PA (US)

(21) Appl. No.: 19/640,628

(22) Filed: Apr. 7, 2026

Related U.S. Application Data
(60) Provisional application No. 63/786,672, filed on Apr. 10, 2025.

(51) Int. Cl. A61K 40/42; A61K 40/11; A61K 40/31; A61P 35/00; C07K 14/705; C07K 16/28; C12N 5/0783; C12N 9/10

(52) U.S. Cl. CPC A61K 40/4211; A61K 40/11; A61K 40/31; A61P 35/00; C07K 14/7051; C07K 16/2803; C12N 5/0636; C12N 9/1081; C12Y 204/99001; C07K 2319/02; C07K 2319/03; C12N 2510/00

(57) ABSTRACT

The subject invention provides engineered chimeric antigen receptor (CAR)-T cells expressing an enzyme to enforce surface α2,6 sialylation and reduce galectin binding. Such engineered CAR-T cells show an improved anti-tumor activity and persistence, which can be used as anti-tumor agents in CAR-T cell therapy for treating cancer or tumors. Also provided are compositions comprising the engineered CAR-T cells and methods of use thereof.

### CROSS-REFERENCE TO RELATED APPLICATION

This application claims the benefit of U.S. Provisional Application Ser. No. 63/786,672, filed Apr. 10, 2025, the disclosure of which is hereby incorporated by reference in its entirety, including all figures, tables, and drawings.

### SEQUENCE LISTING

The Sequence Listing for this application is labeled “SeqList-10Apr25.xml,” which was created on Apr. 10, 2025, and is 2,964 bytes. The entire content is incorporated herein by reference in its entirety.

### BACKGROUND OF THE INVENTION

Diffuse large B cell lymphoma (DLBCL) is one of the most common, non-Hodgkin's lymphomas that is extremely aggressive. DLBCL affects 5.6 out of 100,000 people annually and, sadly, half of DLBCL patients exhibit refractory disease or relapse (r/r) after initial response to standard therapy with a median survival of less than 12 months.

A growing treatment option for (r/r) DLBCL patients is called chimeric antigen receptor (CAR)-T cell therapy. This promising adoptive anti-tumor T cell therapy involves autologous ex vivo gene-modified T cells expressing a CAR that can recognize malignant B cells, eliciting tumoricidal activity. This therapy uses patient-derived T cells that are engineered to recognize a distinct tumor antigen by combining antibody-driven specificity with the activation machinery of the T cell receptor (TCR) complex and co-stimulator activation modules.

However, its efficacy is hindered by unmet challenges, including limited anti-tumor persistence in vivo and therapy-limiting side effects, such as cytokine-release syndrome (CRS) and immune effector cell-associated neurotoxicity syndrome (ICANS). While CAR-T cell therapy has improved patient outcomes, its limited half-life in patients results in a lack of sustained persistence of anti-tumor activity. Moreover, lack of therapy persistence leads to multiple cell infusions and/or higher cell inoculum doses that cause toxic side effects.

There are still ongoing research efforts seeking to design a more efficacious CAR-T cell formulation with the goal of enhancing CAR-T cell tumoricidal activity and in vivo persistence. Boosting these activities could potentially lower the inoculum of CAR-T cells while diminishing therapy-limiting side effects.

Cell surface glycans, referred to as the glycome, are found on proteins and lipids consisting of a single monosaccharide, disaccharides, or more complex oligosaccharides structures. Glycans play a significant regulatory role in immunological homeostasis and inflammation by serving as binding moieties for native glycan-binding proteins (lectins) including β-galactoside-binding galectins, which influence leukocyte homing, adhesion, and apoptosis. Galectins influence several regulatory and signaling pathways that impact T cell fate and function.

Galectins, a family of 15 secreted lectins (12 in humans), are known as potent regulators of leukocyte activity in the immune system (FIG. 1A). Galectins bind β-galactoside glycans with a clear preference for binding specific membrane glycoprotein receptors. In adaptive immunity, studies show that galectin (Gal)-1, -3, and -9 can differentially modulate development, activation, differentiation, and effector T cell function (FIG. 1). Gal-1, -3, and -9 are considered therapeutic targets along with their glycan ligands to overcome immune evasion shortcomings of current immunotherapies. They bind T cells and enable tumor immune escape by activating T cell co-inhibitory receptors, obstructing costimulatory pathways, regulating cell activation and cytokine release, skewing differentiation, and impairing cell survival.

Studies have shown that anti-tumor T cells and their surface glycome are vulnerable to binding host β-galactoside-binding galectin (Gal)-1, -3 and -9, which cause immunosuppressive effects. That is, glycan features on anti-tumor CD4⁺ and CD8⁺ T cells avidly bind Gal-1, -3 and -9 markedly dampen effector cytokine synthesis or cytolytic activity and induce apoptosis. Thus, there is a need to determine surface glycan features of T cells and develop adoptive T cell therapies with an improved efficacy that evade galectin-dependent immunomodulation for cancer treatment.

### BRIEF SUMMARY OF THE INVENTION

The subject invention provides materials, compositions and methods for treating cancer using improved CAR-T cells as anti-tumor agents. In one embodiment, materials, compositions and methods of the subject invention can be used to circumvent galectin binding to the glycome of CAR-T cells and subsequent inhibitory effects, thereby improving the efficacy of CAR-T cell therapy and preventing apoptosis of the CAR-T cells.

The present invention provides strategies to improve CAR-T cell function and persistence for greatly accentuating anti-tumor activity and lowering the cell dose to avoid side effects. By studying glycan and glycosyltransferases in CAR-T cells used in DLBCL therapy as well as other CAR-T cell models with other antigen/co-stimulatory domains to expose galectin-binding capacity, new methods to evade Gal-1/-3-binding and protect CAR-T cells from β-galactoside-binding galectin (e.g., Gal-1 and -3) immunomodulation have been developed.

In one embodiment, the subject invention provides an engineered CAR-T cell expressing a chimeric antigen receptor (CAR) and overexpressing an enzyme having glycosyltransferase activity, the CAR comprising an extracellular domain that specifically binds to a cancer antigen, a transmembrane domain, and at least one intracellular co-stimulatory signaling domain.

In a specific embodiment, the enzyme is α2,6 sialyltransferase (ST6GAL1). In specific embodiments, the engineered CAR-T cell comprises a nucleic acid sequence of ST6GAL1 or a nucleic acid sequence sharing at least 70%, 75%, 80%, 85%, 90%, 95%, 96%, 97%, 98%, 99%, 99.5%, or 99.9% identity with the nucleic acid sequence of ST6GAL1. In a specific embodiment, the engineered CAR-T cells are ST6^{OE} CAR-T cells comprising ST6GAL1/anti-CD19/BBzeta.

In certain embodiments, the engineered CAR-T cells are genetically modified to overexpress an amino acid sequence of SEQ ID NO: 1 or 2, biologically-active fragments, variants thereof, or an amino acid sequence sharing at least 50%, 55%, 60%, 65%, 70%, 75%, 80%, 85%, 90%, 95%, 96%, 97%, 98% or 99% identity with SEQ ID NO: 1 or 2.

In certain embodiments, the cancer antigen is selected from B-cell maturation antigen (BCMA), CD19, CD20, CD22, CD33, CD70, CD123, CD166, mesothelin, CEA, c-Met, PSMA, GD-2, GD-3, FRα, CAIX, CD171, EGFRVIII, HER2, CD133, CEACAM5, CEACAM6, CA-125 (Muc16), sLe^{A} (CA19-9), EGFR, GPC3, PSMA, ROR1, VEGFR2, B7-H3, IL-13Rα, PD-L1, IL-11Rα, EphA2, MAGE, MCAM, NKG2D ligands, TEM1, FAP, GAGE, MUC1, NY-ESO-1, Mesothelin (MSLN), TnMUC1, sialo-Tn (sTn) MUC1 and alpha-folate receptor.

In one embodiment, the subject invention provides a pharmaceutical composition comprising the engineered CAR-T cells of the subject invention. In a specific embodiment, the pharmaceutical composition comprises ST6^{OE} CAR-T cells.

In certain embodiments, the subject invention provides a method for treating cancer in a subject, the method comprising administering to the subject having cancer an effective amount of the engineered CAR-T cells of the subject invention or the pharmaceutical composition of the subject invention. In specific embodiments, the engineered CAR-T cells are adoptive anti-tumor T cells derived from the subject's own lymphocytes.

In one embodiment, the subject invention provides a method for inhibiting galectin-dependent immunosuppression of a CAR-T cell therapy in a subject having cancer, the method comprising administering to the subject having cancer an effective amount of the engineered CAR-T cells of the subject invention or the pharmaceutical composition of the subject invention. In specific embodiments, the galectin is galectin-1, galectin-3, and/or galectin-9.

In some embodiments, the administration is selected from local, intravenous, intradermal, subcutaneous, and intramuscular routes.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1B show Gal-1, -3, and -9 as regulators of anti-tumor T cell immunity. (A) Structural categories of galectins: proto-type, containing one carbohydrate-recognition domain (CRD); chimera-type, with one CRD and an amino-terminal polypeptide tail region that forms Pentamers; and tandem repeat-type with two distinct CRDs. (B) Effects of Gal-1, -3 and -9 on T cell function: (1) Th2 cell skewing; (2) Treg cell expansion, (3) elevation in M2 macrophages; (4) increase in IL-10⁺ T regulatory type 1 (Tr1) cells; and (5) apoptosis of anti-tumor T cells.

FIGS. 2A-2C show that CAR-T cells possess strong Gal-1 and Gal-3 binding activity. Flow cytometry using rhGal-1 (10 μg/ml) (A), rhGal-3 (5 μg/ml) (B), and rhGal-9 (4 μg/ml) (C) and anti-CD4/8 Abs were performed on human naïve T cell MACS-sorted from PBMC (n=6), anti-CD3/28 activated T cells (Day 2), Day 5- or Day 10-expanded T cells, and Day 10-expanded CAR-T cells. Gal-1 and Gal-3 binding to CD4⁺ and CD8⁺ CAR-T cells were significantly higher than to naïve T cells. Addition of negative control 10 mM lactose to rhGal-1, -3, or -9 neutralized galectin binding. Statistical significance—Student's paired t-test.

FIGS. 3A-3C show that CAR-T cells express a conspicuously low level of Gal-1- and Gal-3-binding inhibitor, α2,6-sialyltransferase 1 (ST6GAL1). RT-qPCR analysis on (A) MGAT5, GCNT1, GCNT2, and B4GALT1 and on (B) ST6GAL1 and ST3GAL1 was performed on mRNA from human naïve T cell MACS-sorted from PBMC (n=6), anti-CD3/28 activated T cells (Day 2), Day 5-expanded, Day 10-expanded, and CAR-T cells. Relative Fold Change normalized to GAPDH expression showed that, compared with naïve T cells, glycan-forming enzymes MGAT5 and B4GALT1 in CAR-T cells were elevated (p<0.01 and p<0.01, respectively); whereas, when compared with Day 2 T cells, ST6GAL1 in CAR-T cells was reduced by more than 5-fold (p<0.01). Statistical significance—Student's Paired t-test. In (C), transcriptomic analysis of ST6GAL1, MGAT5 and B4GALT1 was performed on CAR-T cells before and 7 days after infusion in DLBCL patients. CAR-T cells (8 donors) were sorted processed for 10× Genomics, and sequenced by Novogene 6000. Data showed that, compared with pre-infused levels, ST6GAL1 was depressed in post-infused CAR-T cells (p<0.05) and B4GALT1 and MGAT5 were unchanged. Statistical tests—two-sided Mann-Whitney-Wilcoxon test with Bonferroni corrections. (D) Model illustrating how elevated MGAT5/B4GALT1-driven glycan branching and elongation, together with suppressed ST6GAL1 expression, promotes Gal-3 ligand formation. MFIs were background-corrected by subtraction of the appropriate negative control (lactose, isotype, or unstained).

FIG. 4 shows a schematic of CAR-T cells expressing ST6GAL1 (α2,6 sialylation).

FIGS. 5A-5G show anti-CD19 CAR-T cells displayed elevations in tri-antennary N-glycans and poly-N-acetyllactosamines (poly-LacNAc) and low levels of α2,6 sialylation. MALDI-TOF/TOF MS spectra of permethylated N-glycans of (A) CD4⁺ and CD8⁺ naïve T cells, (B) CD4⁺ and CD8⁺ Day 10-expanded T cells and (C) CD4⁺ and CD8⁺ anti-CD19 CAR-T cells. T cell controls and CAR-T cells were stained with plant lectins STA (D), which binds poly-LacNAc, SNA (E), which binds α2,6-linked sialic acids, and MAL-II (F), which binds α2,3-linked sialic acids. Data are graphically presented as MFI±(SEM) on Day 0 (Naïve), Day 2 (activated T cells), Day 5- and Day 10-expanded T cells and CAR-T cells from at least 4 independent donors. Statistical significance was determined using a Kruskal-Wallis test with Dunn's multiple-comparisons correction (*p<0.05).

FIGS. 6A-6I show_ST6^{OE} CAR-T cells exhibited suppressed Gal-1 and -3 ligand activity and resist Gal-3-mediated cell death. Anti-CD19 CAR-T cells and ST6^{OE} CAR-T were stained with rhGal-1 (A) CD4⁺ and (B) CD8⁺ and rhGal-3 (C) CD4⁺ and (D) CD8⁺ to assessed ligand binding via flow cytometry. Data represents n=6 independent donors. Statistical significance was determined using a Mann-Whitney U test (*p<0.05). (E) Gal-3 dose-response analysis showing a concentration-dependent increase in T-cell apoptosis following overnight incubation with increasing concentrations of rhGal-3 (0.5-10 μg/mL), quantified as Annexin V⁺/PI⁺ cells. (F) Activated T cells incubated overnight with rhGal-3 (4 μg/mL) exhibited significantly increased Annexin V⁺/PI⁺ staining compared with lactose-treated controls. (G) Comparison of Gal-3-induced apoptosis between untreated (UT) Day 10-expanded T cells and Day 10 anti-CD19 CAR-T cells following overnight rhGal-3 exposure. (H) Comparison of Gal-3-induced apoptosis between Day 10 anti-CD19 CAR-T cells and ST6^{OE} CAR-T cells following overnight rhGal-3 exposure, demonstrating reduced Annexin V⁺/PI⁺ staining in ST6^{OE} CAR-T cells. For panels (E-H), Annexin V⁺/PI⁺ values are presented as mean±SEM from >4 independent donors, and statistical significance was determined using a Mann-Whitney U (*p<0.05, **p<0.01, ***p<0.001). (I) Baseline proliferation of anti-CD19 CAR-T cells and ST6^{OE} CAR-T cells assessed by CCK-8 assay over 72 h. Data are presented as optical density (OD 450 nm)±SEM from n=4 independent donors and analyzed using a two-way repeated-measures ANOVA with Sidak's multiple-comparisons test (*p<0.001).

FIGS. 7A-7B show that ST6GAL1 can block Gal-1- and Gal-3-induced CAR-T cell death. After incubation for 16 hr with (a) 5 μg/ml rhGal-1 or (b) 4 μg/ml rhGal-3, CAR-T cell and ST6^{OE} CAR-T cell death was assessed by FACS analyzing with Annexin V and Propidium Iodide (PI). Compared with cell death (Annexin V⁺ and PI⁺) in Gal-1- and Gal-3-treated CAR-T cells, ST6^{OE} CAR-T cells were relatively resistant to Gal-1- and Gal-3-induced cell death (p<0.001) (Data from n=4 donors; Student's Paired t-test).

FIGS. 8A-8E show_ST6^{OE} CAR-T cells exhibited potent in vivo anti-tumor activity and persisted longer in vivo than conventional CAR-T cells. Using a DLBCL xenograft model (A), NSG mice were inoculated with Raji-luc⁺ cells and, after 7 days, treated with media (control), Day 10-expanded T cells, CAR-T cells, or ST6^{OE} CAR-T cells. Tumor growth in NSG mice was monitored weekly by in vivo BL imaging system (B), and BLI measurements reflecting tumor burden from respective groups were graphed as shown (n=9 mice per group) (**p<0.01) (C). Xenografted mouse survival data were graphed as shown (Gehan-Breslow-Wilcoxon test—**p=0.0106) (D), and flow cytometry of CAR⁺ T cells isolated from xenografted mouse spleens expressed as Mean±SEM were graphed as shown (Mann-Whitney U test—**p<0.05) (E).

FIG. 9 shows preclinical efficacy of ST6^{OE} CAR-T cells on growth of human B-lymphoma Raji xenografts. NSG mice (8/group) were transplanted i.v. with luc⁺ Raji B-lymphoma cells (10⁵) were treated i.v. with media alone or with 5×10⁶ D10-expanded T cells, CAR-T cells or ST6^{OE} CAR-T cells. (a) Tumor burdens were assayed 2-times/week and graphed as BLI/time. (b) At necropsy, spleens were removed and FACS analyzed for CAR⁺-T cells. Statistical significance, Paired t-test=**p<0.01.

FIG. 10 shows that ST6GAL-1 is a negative regulator of Gal-1/-3-induced CAR-T cell Immunosuppression.

FIGS. 11A-11H show Galectin expression and ligand profiles in DLBCL and CAR-T cells. RNAseq analysis comparing DLBCL patients in remission versus early failure showed no significant differences in (A) LGALS1 (Gal-1), (B) LGALS3 (Gal-3), or (C) LGALS9 (Gal-9) expression. Flow cytometry analysis of galectin binding to CAR-T cells represented by histograms for (D) Gal-1, (E) Gal-3, and (F) Gal-9 ligands compared to isotype Ab alone and lactose-containing controls. (G) SNA lectin staining demonstrated increased α2,6-sialylation in ST6^{OE} CAR-T cells compared to control CAR-T cells (*p<0.05). (H) Gal-3 expression was assessed by flow cytometry and showed higher Gal-3 levels on SUDHL-6 cells than on Raji cells (*p<0.05).

FIGS. 12A-12B show SNA gating strategy for ST6^{oe} CAR-T cells. (A) Gating strategy used to assess SNA lectin binding on ST6^{OE} CAR-T cell. (B) SNA lectin staining demonstrated increased α2,6-sialylation in ST6^{OE} CAR-T cells compared to control CAR-T cells (*p<0.05). Data represent n=4 independent donors. Statistical significance was determined using a Mann-Whitney U test (*p<0.05).

FIGS. 13A-13I show a Gal-3 is elevated in DLBCL patients, DLBCL tumor cells, and DLBCL tumor-infiltrating macrophages. ELISA of serum Gal-1, -3, and -9 levels depicted significantly higher circulating levels of Gal-3 and -9 in DLBCL patients, compared to healthy individuals (***p<0.001 **p<0.01) (A) Gal-1, (B) Gal-3, (C) Gal-9. Transcriptional expression analysis of GEO GSE2350 datasets shows elevated expression of Gal-1 and Gal-3 in DLBCL cells compared to healthy B cell subsets (*p<0.05, ***p<0.001) (D), LGALS1 (E) LGALS3 and (F) LGALS9. Analysis of immune cell infiltrates within DLBCL samples retrieved from TCGA datasets revealed (G) correlations and infiltration score of LGALS3 expression between 24 immune infiltrates. A positive spearman correlation was observed between LGALS3 expression and macrophage infiltration (H) and overall immune infiltration score, whereas a negative correlation was observed between LGALS3 and B cell infiltration (I). Statistical significance was determined using Spearman correlation with false discovery rate (FDR) correction (FDR ≤0.05).

FIG. 14 shows Galectin-3 expression in DLBCL cell lines. Gal-3 expression was assessed by flow cytometry and showed higher Gal-3 levels on SUDHL-6 cells than on Raji cells (*p<0.05). Experiment were repeated 4 times. Statistical significance was determined using a Mann-Whitney U test (*p<0.05).

FIGS. 15A-15H show ST6^{OE} CAR-T cells exhibited effective tumoricidal activity in the presence of Gal-3 and evaded Gal-3-induced expression of IL-5, which compromised anti-tumor efficacy. Day 10-expanded T cells, CAR-T cells, and ST6^{OE} CAR-T cells were incubated with Gal-3^{low} Raji-luc⁺ cells or Gal-3^{high} SUDHL-6-luc⁺ cells at effector:target (E:T) ratios of 20:1. Raji cell cytotoxicity was evaluated (A) without rhGal-3 or (B) with rhGal-3, and (C) Gal-3^{high} SUDHL-6 cell cytotoxicity was examined without exogenous rhGal-3. Data are presented from n=4 independent donors and analyzed using a two-way repeated-measures ANOVA with Sidak's multiple-comparisons test, with **p<0.01, *p<0.05. (D) Human cytokine proteome profiling of supernatants from Day 10-expanded T cells, CAR-T cells, and ST6^{OE} CAR-T cells incubated with rhGal-3 for 16 h revealed a marked upregulation of IL-5 (red hatched box) in CAR-T cells that was absent in ST6^{OE} CAR-T cells. (E) Arrays were repeated 4 times and graphed as Relative Optical Density (Mean±SEM). Intracellular IL-5 expression was assessed by flow cytometry in control Day 10-expanded T cells, CAR-T cells and ST6^{OE} CAR-T cells after rhGal-3 incubation for 16 h and mean±SEM fold change from n=4 independent donors was graphed (F) (**p<0.05). Statistical significance was determined using a Mann-Whitney U test. CAR-T cells and ST6^{OE} CAR-T cells were incubated with (G) Gal-3^{low} Raji-luc⁺ cells or (H) Gal-3^{high} SUDHL6-luc⁺ at E:T ratios of 20:1 (*p<0.05). Statistical significance was determined using two-way repeated-measures ANOVA with Sidak's multiple-comparisons test.

FIGS. 16A-16B show_(A) A cartoon model depicting the impact of Gal-3 on anti-tumor CAR-T cell function and (B) the protective role of enforced ST6GAL1/α2,6 sialylation on Gal-3-dependent CAR-T cell immunoregulation.

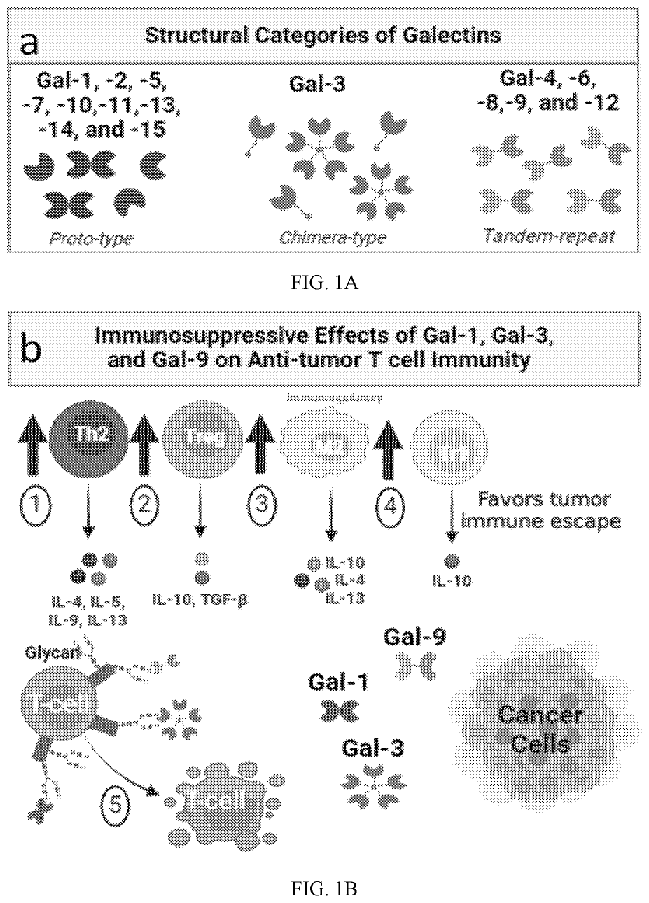

### BRIEF DESCRIPTION OF THE SEQUENCES

SEQ ID NOs: 1 and 2 are amino acid sequences of α2,6 sialyltransferase (ST6GAL1) contemplated for use according to the subject invention.

### DETAILED DESCRIPTION OF THE INVENTION

The subject invention provides materials, compositions and methods for improving CAR-T cell function and persistence to greatly accentuate anti-tumor activity and lower the cell dose to avoid side effects. In one embodiment, materials, compositions and methods of the subject invention can be used to circumvent galectin binding to CAR-T cells and subsequent inhibitory effects, thereby improving the efficacy of CAR-T cell therapy.

In certain embodiments, the subject invention provides materials, compositions and methods for treating cancer or tumor. Specifically, such treatment involves the use of a novel CAR-T cell therapy with engineered CAR-T cells that are superior compared to the existing CAR-T cell therapy. Advantageously, the engineered CAR-T cells are significantly more effective against cancer and tumors, for example, DLBCL, and such engineered CAR-T cells persist longer in vivo.

In a specific embodiment, by studying glycan and glycosyltransferases in CAR-T cells used in DLBCL therapy as well as other CAR-T cell models with other antigen/co-stimulatory domains to expose galectin-binding capacity, the subject invention provides novel methods to evade Gal-1/-3-binding and protect CAR-T cells from Gal-1 and -3 immunomodulation.

In certain embodiments, the subject invention provides a genetic engineering method for producing the engineered CAR-T cells for CAR-T cell therapy. Advantageously, such method is an inexpensive method that can be employed alongside the routine transduction of T cells with the CAR viral construct. This method is a facile approach that can readily be included in the current transduction methods used to make CAR-T cells for adoptive T cell therapies. Further, the technology provided herein is broadly applicable to many adoptive T cell therapies, particularly those used for treating cancer and tumors.

In certain embodiments, the subject invention pertains to Gal-3 levels in DLBCL-associated microenvironments and the glycan landscape of end-of-manufacture anti-CD19 CAR-T cells, focusing on glycosyltransferase expression, Gal-3 ligand availability, and vulnerability to Gal-3-mediated immunoregulation. The subject invention also pertains to glycoengineering CAR-T cells to enforce ST6GAL1 expression in order to mitigate Gal-3-induced dysfunction and enhance CAR-T persistence and antitumor efficacy in a Gal-3-rich tumor microenvironments. The subject invention further provides methods for improving CAR-T cell durability as well as optimized cellular immunotherapies.

### Engineered CAR-T Cells

In one embodiment, the subject invention provides engineered CAR-T cells that target a cancer/tumor antigen. In certain embodiments, the cancer/tumor antigen can be, for example, moieties associated with the cell surface of a cancer cell and is preferably not expressed in normal (non-cancerous) tissues. Suitable cancer antigen targets for CAR T cell therapy include, but are not limited to, BCMA, claudin, CD19, CD20, CD22, CD33, CD70, CD123, CD166, mesothelin, CEA, c-Met, PSMA, GD-2, GD-3, FRα, CAIX, CD171, EGFRVIII, HER2, CD133, CEACAM5, CEACAM6, CA-125 (Muc16), sLe^{A} (CA19-9), EGFR, GPC3, PSMA, ROR1, VEGFR2, B7-H3, IL-13Rα, PD-L1, IL-11Rα, EphA2, MAGE, MCAM, NKG2D ligands, TEM1, FAP, GAGE, MUC1, NY-ESO-1, Mesothelin (MSLN), TnMUC1, sialo-Tn (sTn) MUC1 and alpha-folate receptor. In a specific embodiment, the engineered CAR-T cells recognize CD19 on the cancer or tumor cells, eliciting tumoricidal activity.

In certain embodiments, the engineered CAR-T cells express a CAR and an enzyme having glycosyltransferase activity that catalyzes the addition of sialic acids to the termini of N-glycan antennae, wherein the CAR comprises an extracellular domain that specifically binds to a cancer antigen, and wherein the enzyme is overexpressed compared to normal T cells and/or non-engineered or non-genetically modified CAR-T cells.

In a specific embodiment, the enzyme is α2,6 sialyltransferase (ST6GAL1), variants or fragments thereof. In an exemplary embodiment, ST6GAL1 contains 406 amino acids and has an Accession No. NP_001340845. In a specific embodiment, ST6GAL1 has the following amino acid sequence:

(SEQ ID NO: 1)
MIHTNLKKKFSCCVLVFLLFAVICVWKEKKKGSYYDSFKLQTKE
FQVLKSLGKLAMGSDSQSVSSSSTQDPHRGRQTLGSLRGLAKAK
PEASFQVWNKDSSSKNLIPRLQKIWKNYLSMNKYKVSYKGPGPG
IKFSAEALRCHLRDHVNVSMVEVTDFPFNTSEWEGYLPKESIRT
KAGPWGRCAVVSSAGSLKSSQLGREIDDHDAVLRFNGAPTANFQ
QDVGTKTTIRLMNSQLVTTEKRFLKDSLYNEGILIVWDPSVYHS
DIPKWYQNPDYNFFNNYKTYRKLHPNQPFYILKPQMPWELWDIL
QEISPEEIQPNPPSSGMLGIIIMMTLCDQVDIYEFLPSKRKTDV
CYYYQKFFDSACTMGAYHPLLYEKNLVKHLNQGTDEDIYLLGKA
TLPGFRTIHC.

In another exemplary embodiment, ST6GAL1 is isoform b containing 175 amino acids and has an Accession No. NP_775321. In a specific embodiment, ST6GAL1 has the following amino acid sequence:

(SEQ ID NO: 2)
MNSQLVTTEKRFLKDSLYNEGILIVWDPSVYHSDIPKWYQNPDY
NFFNNYKTYRKLHPNQPFYILKPQMPWELWDILQEISPEEIQPN
PPSSGMLGIIIMMTLCDQVDIYEFLPSKRKTDVCYYYQKFFDSA
CTMGAYHPLLYEKNLVKHLNQGTDEDIYLLGKATLPGFRTIHC

In a specific embodiment, ST6GAL1 gene has GenBank Accession No. NM_173216 or NC_000003.

In a specific embodiment, the engineered CAR-T cells are glycoengineered CAR-T cells that target a cancer/tumor antigen, wherein the engineered CAR-T cells express, on the cell surface, N-glycan having sialic acids at the termini, which blocks the binding of β-galactoside-binding galectin, specifically, Gal-1 and Gal-3, to the surface of CAR-T cells and prevents the apoptosis of CAR-T cells.

In certain embodiments, the engineered CAR-T cells are genetically modified to comprise a nucleic acid sequence of ST6GAL1 or a nucleic acid sequence sharing at least 50%, 55%, 60%, 65%, 70%, 75%, 80%, 85%, 90%, 95%, 96%, 97%, 98% or 99% identity with the nucleic acid sequence that encodes ST6GAL1 to overexpress ST6GAL1 or a polypeptide encoded by the nucleic acid sequence sharing at least 50%, 55%, 60%, 65%, 70%, 75%, 80%, 85%, 90%, 95%, 96%, 97%, 98% or 99% identity with the nucleic acid sequence of ST6GAL1. In certain embodiments, the engineered CAR-T cells overexpress an amino acid sequence of ST6GAL1, biologically-active fragments, variants thereof, or an amino acid sequence sharing at least 50%, 55%, 60%, 65%, 70%, 75%, 80%, 85%, 90%, 95%, 96%, 97%, 98% or 99% identity with ST6GAL1. In specific embodiments, the engineered CAR-T cells overexpress a nucleic acid sequence of ST6GAL1 and/or an amino acid sequence of ST6GAL1.

In certain embodiments, the engineered CAR-T cells are genetically modified to overexpress an amino acid sequence of SEQ ID NO: 1 or 2, biologically-active fragments, variants thereof, or an amino acid sequence sharing at least 50%, 55%, 60%, 65%, 70%, 75%, 80%, 85%, 90%, 95%, 96%, 97%, 98% or 99% identity with SEQ ID NO: 1 or 2.

In certain embodiments, the engineered CAR-T cells are genetically modified to comprise a nucleic acid sequence that encodes the amino acid sequence of SEQ ID NO: 1 or 2, biologically-active fragments, variants thereof, or an amino acid sequence sharing at least 50%, 55%, 60%, 65%, 70%, 75%, 80%, 85%, 90%, 95%, 96%, 97%, 98% or 99% identity with SEQ ID NO: 1 or 2. In certain embodiments, the engineered CAR-T cells are genetically modified to comprise a a nucleic acid sequence sharing at least 50%, 55%, 60%, 65%, 70%, 75%, 80%, 85%, 90%, 95%, 96%, 97%, 98% or 99% identity with the nucleic acid sequence that encodes the amino acid sequence of SEQ ID NO: 1 or 2, biologically-active fragments, variants thereof, or an amino acid sequence sharing at least 50%, 55%, 60%, 65%, 70%, 75%, 80%, 85%, 90%, 95%, 96%, 97%, 98% or 99% identity with SEQ ID NO: 1 or 2.

In certain embodiments, the engineered CAR-T cells comprise a reduced expression of β1,6 N-acetylglucosaminyltransferase 5 (MGAT5), which adds N-acetylglucosamine (GlcNAc) to mannose core to initiate a tri- and tetra-antennae on N-glycans, core 2 β1,6 N-acetyltransferase 1 (GCNT1), which adds GlcNAc to the O-glycan GalNAc core sugar; and/or β1,4 galactosyltransferase 1 (B4GALT1), which transfers galactose (Gal) to the C4 position of GlcNAc to create the hallmark galectin-binding moiety, Galβ1-4GlcNAc1.

“CAR-T cels” refer to a T cell or population thereof, which has been modified through molecular biological methods to express a CAR on the T cell surface. “Chimeric antigen receptor” or “CAR” refers to an artificial immune cell receptor that is engineered to recognize and bind to a selected cancer/tumor antigen. The CAR is a polypeptide having a pre-defined binding specificity to a desired target expressed operably connected to the intracellular part of a T-cell activation domain. Specifically, the CAR is different from the TCR expressed in the native T cell lymphocyte.

Generally, a CAR is designed for a T cell and is a chimera of a signaling domain of the T cell receptor (TCR) complex (e.g., CD3zeta) and an antigen-recognizing domain (e.g., a single chain fragment (scFv) of an antibody or other antibody fragment). In certain embodiments, CARs comprise an additional domain, e.g., CD28, OX40, CD27, CD2, CD5, ICAM-1, LFA-1, 4-1BB (41BB), or ICOS, to supply a costimulatory signal. In further embodiments, CARs comprises two costimulatory domains selected from, for example, CD3zeta, CD27, CD28, CD2, CD5, ICAM-1, LFA-1, 4-1BB, ICOS, and OX40.

In some embodiments, CARs comprise an extracellular domain, a hinge, a transmembrane domain, and an intracellular domain with one, two, or three signaling domains derived from, for example, CD3 and/or costimulatory molecules. The extracellular domains of CARs are often derived from immunoglobulins, peptides or polypeptides, or fragments thereof, capable of specifically binding an antigen or epitope.

In certain embodiments, the transmembrane domain can be derived from the protein contributing the specific extracellular domain, the protein contributing the signaling domain, the protein contributing the proliferation signaling portion, or a totally different protein. For example, the transmembrane domain can be naturally associated with one of the domains of CAR. In general, any transmembrane region sufficient to anchor the CAR in the membrane can be used. In some embodiments, the transmembrane domain is derived from the ζ, η or FcεR1γ chains. Examples of suitable transmembrane regions for use with the invention include, but are not limited to, the constant (Fc) regions of immunoglobins, human CD8a, and artificial linkers that serve to move the targeting moiety away from the cell surface for improved access to and binding on target cells.

In specific embodiments, the hinge and the transmembrane domain are associated together and from the same protein.

In certain embodiments, the intracellular domain of CAR comprises a signaling domain (e.g., co-stimulatory signaling domain) by itself or combined with any other desired cytoplasmic domain(s) useful in the CAR, for example, a 4-1BB signaling domain, a CD3ζ signaling domain, a CD28 signaling domain and/or any combination thereof. The 4-1BB, CD3ζ and CD28 signaling domains are well characterized in the art, including for example, their use in chimeric receptors. In some embodiments, the intracellular domain of CAR comprises the 4-1BB signaling domain by itself or combined with any other desired cytoplasmic domain(s) useful in the CAR.

In a specific embodiment, the CAR comprises 1) an extracellular domain comprising a single chain variable domain of a monoclonal antibody, 2) a hinge comprising the hinge of CD8α, 3) a transmembrane domain comprising the transmembrane domain of CD8α, and 4) an intracellular domain comprising the signaling domain of CD3ζ and the signaling domain of 4-1BB. For example, the CD3ζ signaling domain contains 112 amino acids translated from 339 nucleotides at positions 1022-1360 of GenBank Accession No. NM_000734.

In specific embodiments, the CAR comprises an extracellular domain that specifically binds CD19 on the cancer to tumor cells. and BBzeta that comprises a fusion of the signaling domain of CD3ζ and the signaling domain of 4-1BB.

In s specific embodiments, the engineered CAR-T cells of the subject invention are ST6^{OE} CAR-T cells. In a specific embodiment, the ST6^{OE} CAR-T cells comprise ST6GAL1/anti-CD19/BBzeta. In a specific embodiment, the engineered CAR-T cells are anti-CD19 CAR-T cells (CD19 scFv/4-1BB/CD3ζ) or ST6GAL1/anti-CD19 CAR-T cells (ST6GAL1-P2A-CD19 scFv/4-1BB/CD3ζ).

In specific embodiments, the engineered CAR-T cells express CARs that target cancer or tumor cells expressing the cancer/tumor antigen and overexpress an enzyme having the glycosyltransferase activity (e.g., ST6GAL1) that blocks the binding of β-galactoside-binding galectin, specifically, Gal-1 and Gal-3, to glycome on the surface of CAR-T cells, preventing the apoptosis of CAR-T cells.

### Compositions

In one embodiment, the subject invention provides a therapeutic/pharmaceutical composition comprising the engineered CAR-T cells of the subject invention and optionally a pharmaceutically acceptable carrier. In specific embodiments, the therapeutic/pharmaceutical composition of the subject invention, when administered in a host or subject, induces an anti-cancer or anti-tumor response, which includes, but are not limited to, specific immune response, non-specific immune response, innate response, primary immune response, adaptive immunity, secondary immune response, memory immune response, immune cell activation, immune cell proliferation, immune cell differentiation, cytokine expression and any combination thereof.

In certain embodiments, the therapeutic/pharmaceutical composition of the subject invention comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

In one embodiment, the therapeutic/pharmaceutical composition of the subject invention can be administered to a subject in an effective amount that provides a therapeutic benefit. Effective amounts of engineered CAR-T cells can be determined by a skilled in the art with consideration of individual differences in age, weight, tumor size, extent of infection or metastasis, and condition of the subject. An effective amount can be one that decrease or ameliorate the symptoms by at least 10%, at least 20%, at least 30%, at least 40%, at least 50%, at least 60%, at least 70%, at least 80%, at least 90%, at least 95%, at least 99%, or at least 99.9%.

In certain embodiments, the therapeutic/pharmaceutical composition can be administered at a dosage of about 10² to 10¹¹ cells/kg body weight, 10³ to 10¹¹ cells/kg body weight, 10⁴ to 10¹¹ cells/kg body weight, 10⁵ to 10¹¹ cells/kg body weight, 10⁶ to 10¹¹ cells/kg body weight, 10⁷ to 10¹¹ cells/kg body weight, 10⁸ to 10¹¹ cells/kg body weight, 10⁹ to 10¹¹ cells/kg body weight, 10⁴ to 10¹⁰ cells/kg body weight, 10⁵ to 10¹⁰ cells/kg body weight, 10⁶ to 10¹⁰ cells/kg body weight, 10⁷ to 10¹⁰ cells/kg body weight, 10⁸ to 10¹⁰ cells/kg body weight, 10⁴ to 10⁸ cells/kg body weight, 10⁴ to 10⁷ cells/kg body weight, 10³ to 10⁷ cells/kg body weight, 10² to 10⁶ cells/kg body weight, 10² to 10⁵ cells/kg body weight, 10² to 10⁴ cells/kg body weight, 10³ to 10⁵ cells/kg body weight, or 10³ to 10⁴ cells/kg body weight.

In certain embodiments, the therapeutic/pharmaceutical compositions may also be administered in one dose, two doses or multiple times at these dosages. Where more than one dose is administered, the therapeutic/pharmaceutical composition can be administered with time intervals of at least 1 minute, 5 minutes, 10 minutes, 15 minutes, 20 minutes, 30 minutes, or more, or time intervals of at least about 1 hour, 2 hours, 5 hours 8 hours, 10 hours, 12 hours, 16 hours, 20 hours, 24 hours, or 48 hours. In certain embodiments, the therapeutic/pharmaceutical composition can be administered with time intervals of at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In certain embodiments, the therapeutic/pharmaceutical composition can be administered, for example, once/week, twice/week, three times/week, four times/week, five times/week, six times/week, seven times/week, once every two weeks, once every three weeks, once every four weeks, once every five weeks, and the like. In certain embodiments, the therapeutic/pharmaceutical composition can be administered for a total period of time of at least, for example, one week, two weeks, three weeks, four weeks, five weeks, six weeks, two months, three months, four months, five months, six months, seven months, eight months, nine months, ten months, eleven months, and twelve months.

In certain embodiments, the therapeutic/pharmaceutical compositions can be suitable for administration by any acceptable route, including parenteral and subcutaneous. Other routes include, for example, intravenous, intradermal, intramuscular, intraperitoneal, intranodal and intrasplenic administration. In specific embodiments, the therapeutic/pharmaceutical composition is suitable for systemic (e.g., intravenous) administration. If the composition is in a form intended for administration to a subject, it can be made to be isotonic with the intended site of administration. For example, if the solution is in a form intended for administration parenterally, it can be isotonic with blood. The composition typically is sterile. In certain embodiments, parenteral compositions generally are placed into a container having a sterile access port, for example, an intravenous solution bag, or vial having a stopper pierceable by a hypodermic injection needle, or a prefilled syringe. In a specific embodiment, the composition can be administered by using infusion techniques that are commonly known in immunotherapy.

In some embodiments, the therapeutic/pharmaceutical composition of the subject invention can be co-administered with one or more other therapeutic agents for treating cancer. In specific embodiments, the other therapeutic agent is a chemotherapeutic agent selected from, for example, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, melphalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, 5-fluorouracil, and the like.

In certain embodiments, the other therapeutic agent is an immune checkpoint inhibitor (ICI) that decreases, blocks, inhibits, abrogates or interferes with the function of a protein of an immune checkpoint pathway. In certain embodiments, the protein of the immune checkpoint pathway is, for example, CTLA-4, PD-1, PD-L1, PD-L2, B7-H3, B7-H4, TIGIT, VISTA, LAG3, CD112 TIM3, BTLA. In certain embodiments, the ICI is a small molecule, an inhibitory nucleic acid, or an inhibitor polypeptide. In various aspects, the ICI is an antibody, antigen-binding antibody fragment, or an antibody protein product, that binds to and inhibits the function of the protein of the immune checkpoint pathway. Suitable ICIs which are antibodies, antigen-binding antibody fragments, or an antibody protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-L1; Merck), and durvalumab (PD-L1; Medimmune). Other examples of ICIs include, but are not limited to, IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); MGA271 (B7-H3: MacroGenics); and TSR-022 (TIM3; Tesaro).

### Glycoengineering

In one embodiment, the subject invention provides a method for engineering CAR-T cells to circumvent the binding of β-galactoside-binding galectin, e.g., Gal-1, -3, or -9 and related inhibitory effects. In one embodiment, the subject invention provides a method for preparing engineered CAR-T cells that are genetically modified to overexpression an enzyme having the glycosyltransferase activity that catalyzes the addition of sialic acids to the termini of N-glycan antennae.

In certain embodiments, the method can be accomplished through the introduction of an enzyme encoding gene into the genomic nucleic acid of CAR-T cells followed by expansion in vitro to arrive at stable CAR-T cells expressing the enzyme having the glycosyltransferase activity. In a specific embodiment, the enzyme is ST6GAL1.

In a specific embodiment, the subject invention provides a method for preparing the glycoengineered CAR-T cells that target a cancer/tumor antigen (e.g., CD19), wherein the glycoengineered CAR-T cells express, on the cell surface, N-glycan having sialic acids at the termini.

In certain embodiments, the subject invention provides genetic modifications for introduction of the CAR construct into T cells, which can be accomplished by transducing (or otherwise delivering) T cells with one or more recombinant DNA or RNA constructs, for example, vectors, that comprise a nucleic acid sequence encoding the CAR, the enzyme having the glycosyltransferase activity, or a combination thereof.

In some embodiments, a vector can be any agent capable of delivering or maintaining nucleic acid in a host cell. Suitable vectors include viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate sequence (e.g., DNA) can be inserted into the vector by a variety of procedures, for example, into an appropriate restriction endonuclease site(s) by procedures known in the art. Such procedures are deemed to be within the scope of those skilled in the art.

In a preferred embodiment, a retroviral vector (e.g., gamma-retroviral and lentiviral) is employed for the introduction of the CAR nucleic acid construct into the cell. For example, a polynucleotide encoding a co-stimulatory ligand protein (e.g., tumor necrosis factor (TNF) ligand, such as 4-1BBL, OX40L, CD70, LIGHT, and CD30L, or an Ig superfamily ligand, or a receptor that binds an antigen, or a variant, or a fragment thereof, can be cloned into a retroviral vector and expression can be driven from its endogenous promoter, from the retroviral long terminal repeat, or from a promoter specific for a target cell. Non-viral vectors may be used as well.

In certain embodiments, the subject invention provides a method for producing the engineered CAR-T cells of the subject invention, the method comprising:
producing/providing a vector comprising a nucleic acid sequence encoding a CAR, an enzyme having the glycosyltransferase activity, or a combination thereof;
providing T cells, preferably, the T cells being activated T cells; and
transducing said T cells with said vector to express the CAR and overexpress the enzyme having the glycosyltransferase activity in the T cells.

In certain embodiments, the method of the subject invention further comprises determining the expression of the enzyme having the glycosyltransferase activity in the engineered CAR-T cells; and determining the expression of CAR in the engineered CAR-T cells.

In certain embodiments, the method of the subject invention further comprises transducing the T cells with a vector comprising a nucleic acid sequence encoding a CAR to produce a CAR-T cells, wherein said vector does not comprise a nucleic acid sequence encoding an enzyme having the glycosyltransferase activity. In certain embodiments, the method of the subject invention further comprises comparing the expression of the enzyme having the glycosyltransferase activity in the engineered CAR-T cells to the expression of said enzyme in the T cells and/or the CAR-T cells that have not been genetically modified to overexpress the enzyme having the glycosyltransferase activity.

In certain embodiments, the step of producing the vector comprising a nucleic acid sequence encoding a CAR, an enzyme having the glycosyltransferase activity, or a combination thereof comprises providing a host cell; transducing the host cell with one or more expression constructs; and collecting the vector, wherein the one or more expression constructs comprise comprising a nucleic acid sequence encoding a CAR, an enzyme having the glycosyltransferase activity, or a combination thereof. In specific embodiments, the host cell is a human cell or bacterial cell.

Expression constructs of the invention generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements. An expression construction preferably also includes sequences to allow for the replication of the expression construct. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

In certain embodiments, promoters can be selected from, for example, cell specific promoters (e.g., human CD2, distal Lck, and proximal Lck), non-tissue specific promoters (e.g., viral promoters such as cytomegalovirus (CMV) promoter, β-actin promoter phosphoglycerate kinase (PGK) promoter, ubiquitin promoter, and EF-1α promoter). In certain embodiments, enhancers that act on a promoter to increase its transcription include, for example, the SV40 enhancer, a cytomegalovirus early promoter enhancer, the polyoma enhancer, and adenovirus enhancers.

In specific embodiments, the one or more expression constructs are selected from anti-CD19 CAR plasmids, dual CD19 CAR and ST6GAL1 expressing CAR plasmids, and lentiviral packaging plasmids.

In certain embodiments, the step of providing T cells comprises collecting a sample (e.g., blood or tumor sample) from a subject; isolating lymphocytes (e.g., circulating lymphocytes or tumor infiltrated lymphocytes) from the sample; and optionally, treating the isolated lymphocytes with a lymphokines such as IL-2 to arrive at activated T cells. In preferred embodiments, the isolated lymphocytes and activated lymphocytes are the subject's own cells that were earlier isolated from a blood or tumor sample and activated and expanded in vitro. The engineered CAR-T cells can be expanded in vitro for use in adoptive cellular immunotherapy in which infusions of such cells has anti-tumor reactivity in the subject having cancer or tumor.

In certain embodiment, the sample is selected from, for example, broncho-alveolar lavage fluid (BALF) circulation fluids, secretion fluids, biopsy, and homogenized tissue, particularly blood, serum, urine, tear, saliva, bile, sweat, exhalation or expiration, sputum, bronchoalveolar fluid, sebum, cellular, gland, mucosa and tissue secretion. In certain embodiments, the sample may be a non-diluted or diluted biological fluid, such as, without being restricted thereto, blood, serum, urine, tear, saliva, bile, sweat, exhalation or expiration, sputum, bronchoalveolar fluid, sebum, cellular, gland, mucosa or tissue secretion, biopsy, or homogenized tissue.

In certain embodiments, the vector used for transducing the T cells comprises a nucleic acid sequence encoding CAR of the subject invention; and/or a nucleic acid sequence encoding an enzyme having the glycosyltransferase activity. In specific embodiments, the vector comprises a nucleic acid sequence of ST6GAL1; a nucleic acid sequence sharing at least 50%, 55%, 60%, 65%, 70%, 75%, 80%, 85%, 90%, 95%, 96%, 97%, 98% or 99% identity with the nucleic acid sequence that encodes ST6GAL1; or a nucleic acid sequence encoding a protein having an amino acid sequence sharing at least 50%, 55%, 60%, 65%, 70%, 75%, 80%, 85%, 90%, 95%, 96%, 97%, 98% or 99% identity with ST6GAL1 and having the glycosyltransferase activity. In a specific embodiment, the vector comprises a nucleic acid sequence encoding ST6GAL1.

In certain embodiments, the vector comprises a nucleic acid sequence that encodes the amino acid sequence of SEQ ID NO: 1 or 2, biologically-active fragments, variants thereof, or an amino acid sequence sharing at least 50%, 55%, 60%, 65%, 70%, 75%, 80%, 85%, 90%, 95%, 96%, 97%, 98% or 99% identity with SEQ ID NO: 1 or 2.

In a specific embodiment, the vector is a lentivirus vector comprising ST6GAL1/CD19/BBzeta.

### Methods of Use

In one embodiment, the subject invention provides the use of the engineered CAR-T cells as anti-tumor agents, and the use of the composition comprising the engineered CAR-T cells for treating cancer.

In one embodiment, the subject invention provides a CAR-T cells therapy as a treatment option for cancer, in particular, (r/r) DLBCL. In specific embodiments, the CAR-T cell therapy is an adoptive anti-tumor T cell therapy consists of autologous ex vivo gene-modified T cells expressing a CD19 CAR that can recognize, for example, malignant B cells, eliciting tumoricidal activity and overexpressing an enzyme having the glycosyltransferase activity (e.g., ST6GAL1). This therapy uses patient-derived T cells that are engineered to recognize a distinct tumor antigen, notably CD19, by combining antibody-driven specificity with the activation machinery of the T cell receptor (TCR) complex and co-stimulator activation modules.

In one embodiment, the subject invention provides a method for treating a cancer or tumor in a subject in need of such treatment, the method comprising administering to the subject having cancer the engineered CAR-T cells of the subject invention or the therapeutic composition comprising the engineered CAR-T cells of the subject invention.

In one embodiment, the subject invention provides a method of CAR-T cell therapy for treating cancer in a subject, the method comprising administering to the subject having cancer the engineered CAR-T cells of the subject invention or the therapeutic composition comprising the engineered CAR-T cells of the subject invention.

In certain embodiments, the engineered CAR-T cells overexpress a nucleic acid sequence of ST6GAL1 or a nucleic acid sequence sharing at least 50%, 55%, 60%, 65%, 70%, 75%, 80%, 85%, 90%, 95%, 96%, 97%, 98% or 99% identity with the nucleic acid sequence that encodes ST6GAL1. In certain embodiments, the engineered CAR-T cells overexpress an amino acid sequence of ST6GAL1, biologically-active fragments, variants thereof, or an amino acid sequence sharing at least 50%, 55%, 60%, 65%, 70%, 75%, 80%, 85%, 90%, 95%, 96%, 97%, 98% or 99% identity with ST6GAL1. In specific embodiments, the engineered CAR-T cells overexpress a nucleic acid sequence of ST6GAL1 and/or an amino acid sequence of ST6GAL1.

In certain embodiments, the engineered CAR-T cells comprise a nucleic acid sequence that encodes the amino acid sequence of SEQ ID NO: 1 or 2, biologically-active fragments, variants thereof, or an amino acid sequence sharing at least 50%, 55%, 60%, 65%, 70%, 75%, 80%, 85%, 90%, 95%, 96%, 97%, 98% or 99% identity with SEQ ID NO: 1 or 2.

In certain embodiments, the engineered CAR-T cells overexpress an amino acid sequence of SEQ ID NO: 1 or 2, biologically-active fragments, variants thereof, or an amino acid sequence sharing at least 50%, 55%, 60%, 65%, 70%, 75%, 80%, 85%, 90%, 95%, 96%, 97%, 98% or 99% identity with SEQ ID NO: 1 or 2.

In certain embodiments, the engineered CAR-T cells comprise a reduced expression of β1,6 N-acetylglucosaminyltransferase 5 (MGAT5), core 2 β1,6 N-acetyltransferase 1 (GCNT1), and/or β1,4 galactosyltransferase 1 (B4GALT1).

In certain embodiments, the cancer or tumor may be of any kind as long as it has a cell surface antigen that can be recognized by the CAR. In certain embodiments, the cancer is alveolar rhabdomyosarcoma, bone cancer, brain cancer (e.g., glioblastoma, medulloblastoma, glioma), breast cancer (e.g., triple negative breast cancer), chronic myeloid cancer, colon cancer, esophageal cancer, cervical cancer, gastrointestinal cancer (e.g., gastrointestinal carcinoid tumor), endometrial or hepatocellular carcinoma, hypopharynx cancer, kidney cancer, larynx cancer, liver cancer, lung cancer (e.g., non-small cell lung cancer, bronchioloalveolar carcinoma), malignant mesothelioma, melanoma, multiple myeloma, nasopharynx cancer, ovarian cancer, osteosarcoma, pancreatic cancer, mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

In certain embodiments, the cancer is a hematologic cancer selected from, for example, chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma (DLBCL), follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and preleukemia. In a specific embodiment, the cancer is DLBCL.

In some embodiments, the cancer or tumor can be in any of a variety of stages including newly diagnosed, relapsed, refractory, progressive disease, remission, and others. In some embodiments, the cancer or tumor being treated is a newly diagnosed cancer. In some embodiments, the cancer or tumor is a recurrent cancer. In some embodiments, the present invention is applicable to treatment of metastatic cancers.

In certain embodiments, the engineered CAR-T cells and the therapeutic composition can be utilized in conjunction with other types of therapy for cancer, such as chemotherapy, surgery, radiation, and gene therapy.

In some embodiments, the method of the subject invention further comprises administering one or more other therapeutic agents. In specific embodiments, the other therapeutic agent is a chemotherapeutic agent selected from, for example, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, melphalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, 5-fluorouracil, and the like.

In certain embodiments, the other therapeutic agent is an immune checkpoint inhibitor (ICI) of CTLA-4, PD-1, PD-L1, PD-L2, B7-H3, B7-H4, TIGIT, VISTA, LAG3, CD112 TIM3, BTLA. In certain embodiments, the ICI is a small molecule, an inhibitory nucleic acid, or an inhibitor polypeptide. In various aspects, the ICI is an antibody, antigen-binding antibody fragment, or an antibody protein product, that binds to and inhibits the function of the protein of the immune checkpoint pathway selected from, for example, CTLA-4, PD-1, PD-L1, PD-L2, B7-H3, B7-H4, TIGIT, VISTA, LAG3, CD112 TIM3, BTLA. Suitable ICIs which are antibodies, antigen-binding antibody fragments, or an antibody protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-L1; Merck), and durvalumab (PD-L1; Medimmune). Other examples of ICIs include, but are not limited to, IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); MGA271 (B7-H3: MacroGenics); and TSR-022 (TIM3; Tesaro).

In one embodiment, the subject invention provides a method for targeted killing of cancer or tumor cells in a subject having cancer or tumor, the method comprising administering to the subject having cancer or tumor the engineered CAR-T cells of the subject invention or the therapeutic composition comprising the engineered CAR-T cells of the subject invention. In specific embodiments, the engineered CAR-T cells express CARs that target cancer or tumor cells and overexpress an enzyme having the glycosyltransferase activity (e.g., ST6GAL1) that blocks the binding of Gal-1, -3, and/or -9 to glycome on the surface of CAR-T cells.

In certain embodiments, the cancer or tumor cells are selected from, for example, prostate cancer cells, gallbladder cancer cells, intrahepatic biliary tract cancer cells, biliary tract cancer cells, oral cancer cells, pharyngeal cancer cells, laryngeal cancer cells, tongue cancer cells, duodenal cancer cells, eye tumor cells, mediastinal cancer cells, sinus cancer cells, renal pelvic cancer cells, heart cancer cells, glioblastoma cells, neuroblastoma cells, liver cancer cells, bone cancer cells, pancreatic cancer cells, skin cancer cells, head and neck cancer cells, breast cancer cells, lung cancer cells, skin or intraocular malignant melanoma cells, kidney cancer cells, uterine cancer cells, ovarian cancer cells, colon cancer cells, rectal cancer cells, anal region cancer cells, colorectal cancer cells, stomach cancer cells, testicular cancer cells, fallopian tube cancer endometrial carcinoma cells, cervical carcinoma cells, vaginal carcinoma cells, vulvar cancer cells, non-Hodgkin's lymphoma cells, esophageal cancer cells, small intestinal cancer cells, endocrine system cancer cells, thyroid cancer cells, parathyroid cancer cells, adrenal gland cancer cells, soft tissue sarcoma cells, urethral cancer cells, penile cancer cells, childhood cancer cells, lymphocytic lymphoma cells, bladder cancer cells, ureter cancer cells, renal pelvic carcinoma cells, central nervous system (CNS) cancer cells, primary CNS lymphoma cells, spinal cancer cells, brainstem glioma cells, pituitary adenoma cells, Kaposi's sarcoma cells, epidermal cancer cells, squamous cell carcinoma cells, multiple myeloma cells, B-cell lymphoma cells, Hodgkin's lymphoma cells, acute myeloid lymphoma cells, chronic myeloid leukemia cells, chronic lymphocytic leukemia cells, follicular lymphoma cells, diffuse large B-cell lymphoma cells, Burkitt's lymphoma cells, immune large-cell lymphoma cells, progenitor B-lymphoblastic lymphoma cells, mantle cell lymphoma cells, acute lymphoblastic lymphoma cells, mycosis fungoides cells, anaplastic large-cell lymphoma cells, acute myeloid leukemia cells, acute lymphoblastic leukemia cells, hepatoblastoma cells, retinoblastoma cells, peritoneal cancer cells, brain tumor cells, thymic cancer cells, T-cell lymphoma or precursor T-lymphoblastic lymphoma cells, and any combination of the above cancer cells. In a specific embodiment, the cancer cells are malignant B cells.

In one embodiment, the subject invention provides a method for reducing the viability of cancer or tumor cells in a subject having cancer or tumor, the method comprising administering to the subject having cancer or tumor the engineered CAR-T cells of the subject invention or the therapeutic composition comprising the engineered CAR-T cells of the subject invention.

In one embodiment, the subject invention provides a method for inhibiting the proliferation or growth of cancer or tumor cells in a subject having cancer or tumor, the method comprising administering to the subject having cancer or tumor the engineered CAR-T cells of the subject invention or the therapeutic composition comprising the engineered CAR-T cells of the subject invention.

In one embodiment, the subject invention provides a method for improving efficacy of a CAR-T cell therapy in a subject having cancer or tumor, the method comprising administering to the subject having cancer or tumor the engineered CAR-T cells of the subject invention or the therapeutic composition comprising the engineered CAR-T cells of the subject invention.

In certain embodiments, the engineered CAR-T cells of the subject invention or the therapeutic composition comprising the engineered CAR-T cells can be administered to the subject being treated by standard routes, including, for example, the local, intratumoural, transdermal, intra-articular, parenteral (e.g., intravenous, intraperitoneal, intradermal, subcutaneous or intramuscular), intracranial, or intracerebral. Additionally, the composition may be administered directly into the tumor.

In one embodiment, the subject invention provides a method for inhibiting galectin-dependent immunosuppression of an adoptive T cell therapy (e.g., a CAR-T cell therapy) in a subject having cancer or tumor, wherein the adoptive T cell therapy (e.g., a CAR-T cell therapy) is used to treat the cancer or tumor, the method comprising administering to the subject having cancer or tumor the engineered CAR-T cells of the subject invention or the therapeutic composition comprising the engineered CAR-T cells of the subject invention. In specific embodiments, the galectin is galectin-1, galectin-3, and/or galectin-9.

In certain embodiments, the method of the subject invention further comprises evaluating the growth of the cancer cells or size of the tumor; monitoring the tumor growth; and/or assessing the level of the engineered CAR-T cells, for example, in a sample obtained from the subject.

In one embodiment, the subject invention provides a method for protecting T cells or CAR-T cells from immunomodulation (e.g., immunosuppression) by β-galactoside-binding galectin (e.g., Gal-1, -3, and -9), the method comprising providing CAR-T cells; transducing said CAR-T cells with a vector comprising a nucleic acid sequence encoding an enzyme having the glycosyltransferase activity; and optionally, determining the expression of the enzyme having the glycosyltransferase activity in the CAR-T cells.

In one embodiment, the subject invention provides a method for improving T cells or CAR-T cells function and/or persistence, the method comprising providing CAR-T cells; transducing said CAR-T cells with a vector comprising a nucleic acid sequence encoding an enzyme having the glycosyltransferase activity; and optionally, determining the expression of the enzyme having the glycosyltransferase activity in the CAR-T cells.

In one embodiment, the subject invention provides a method for reducing apoptosis of T cells or CAR-T cells, the method comprising providing CAR-T cells; transducing said CAR-T cells with a vector comprising a nucleic acid sequence encoding an enzyme having the glycosyltransferase activity; and optionally, determining the expression of the enzyme having the glycosyltransferase activity in the CAR-T cells.

In one embodiment, the subject invention provides a method for increasing α2,6 sialylation on the surface of T cells or CAR-T cell, the method comprising providing CAR-T cells; transducing said CAR-T cells with a vector comprising a nucleic acid sequence encoding an enzyme having the glycosyltransferase activity; and optionally, determining the expression of the enzyme having the glycosyltransferase activity in the CAR-T cells.

In a specific embodiment, the enzyme is ST6GAL-1. Advantageously, enforced ST6GAL1 gene expression can enhance CAR-T cell persistence and anti-tumor efficacy, improving CAR-T cell therapy outcomes in not only DLBCL patients but in CAR-T cell therapies against other cancers

### Definitions

To facilitate an understanding of the present invention, a number of terms and phrases are defined below. The terminology used herein is used only for the purpose of describing particular embodiments of the present invention and is not intended to be limiting in any way.

“Pharmaceutically acceptable carrier” refers to a diluent, adjuvant or excipient with which the antigen disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith. A “pharmaceutically acceptable carrier” can include, but is not limited to, sterile distilled water, saline, phosphate buffered solutions, amino acid-based buffers, or bicarbonate buffered solutions.

As used herein, the term “treatment” or any grammatical variation thereof (e.g., treat, treating, treated, etc.) includes but is not limited to, the application or administration to a subject (or application or administration to a cell or tissue from a subject) with the purpose of delaying, slowing, stabilizing, curing, healing, alleviating, relieving, altering, remedying, less worsening, ameliorating, improving, or affecting the disease or condition, or the symptom of the disease or condition. The term “treating” refers to any indication of success in the treatment or amelioration of a pathology or condition, including, but not limited to abatement, remission, lessening of the rate of worsening, lessening severity of the disease, and diminishing of symptoms.

A “nucleic acid” according to the invention refers to polynucleotides, such as DNA, RNA, modified DNA, modified RNA as well as mixtures thereof.

As used herein, “variants” of a protein refer to sequences that have one or more amino acid substitutions, deletions, additions, or insertions. In preferred embodiments, these substitutions, deletions, additions or insertions do not materially adversely affect the protein activity. Variants that retain one or more biological activities are within the scope of the present invention.

“Fragments” and its variants are also within the scope of proteins of the subject invention, so long as the fragment retains one or more biological properties. Preferably, the fragment is at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the full-length protein.

The term “sample” as used herein refers to any physical sample that includes a cell, a tissue, a biofluid or an organ including a biopsy sample. The sample can be from a biological source such as a subject, or a portion thereof, or can be from a cell culture. Samples from a biological source can be from a normal or an abnormal organism, such as an organism known to be suffering from a condition or a disease state, or any portion thereof. Samples can also be from any fluid, tissue or organ including normal and abnormal (diseased) fluid, tissue or organ. Samples from a subject can be used, processed or cultured such that cells from the sample can be sustained in vitro as a primary or continuous cell culture or cell line. In certain embodiments, the fluid encompasses, e.g., serum, plasma, blood, interstitial fluid, cerebrospinal fluid, secreted fluids, lymph, bile, sweat, fecal matter, and urine. In specific embodiments, the fluid can comprise a colloid or a suspension, e.g., whole blood or coagulated blood.

The term “subject” or “patient,” as used herein, describes an organism, including mammals such as primates, to which diagnosis, prevention, assessment, and/or treatment according to the present invention can be provided. Mammalian species that can benefit from the disclosed methods of treatment include, but are not limited to, apes, chimpanzees, orangutans, humans, monkeys; domesticated animals such as dogs, cats; live-stocks such as horses, cattle, pigs, sheep, goats, and chickens; and other animals such as mice, rats, guinea pigs, and hamsters.

“Overexpression” refers to an increase in expression level by at least 10%, at least 20%, at least 30%, at least 40%, at least 50%, at least 60%, at least 70%, at least 80%, at least 90%, at least 95%, at least 99%, or at least 99.9% compared to a reference level, which can be, for example, the expression level before any modification. “Overexpression” also refers to an increase in expression level by at least 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 folds compared to a reference level, which can be, for example, the expression level before any modification.

The term “reduce,” “reducing” or “reduction” refers to a decrease by at least 10%, at least 20%, at least 30%, at least 40%, at least 50%, at least 60%, at least 70%, at least 80%, at least 90%, at least 95%, at least 99%, or at least 99.9% compared to a reference level, which can be, for example, the expression level before any modification. These Terms also refer to a decrease by at least 1.5, 2, 2.5, 3, 3.5, 4, 4.5, 5, 5.5, 6, 6.5, 7, 7.5, 8, 8.5, 9, 9.5, or 10 folds compared to a reference level, which can be, for example, the expression level before any modification.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, Fd, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgGl, IgG2, IgG3, IgG4, IgAQl and IgA2), based on the identity of their heavy-chain constant domains referred to as alpha, delta, epsilon, gamma, and mu, respectively.

The term “antibody fragment” refers to a portion of an intact antibody and refers to the antigenic determining variable regions of an intact antibody. Examples of antibody fragments include, but are not limited to Fab, Fab′, F(ab′)2, Fd, and Fv fragments, linear antibodies, single chain antibodies, and multispecific antibodies formed from antibody fragments.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

Except in the examples, or where otherwise expressly indicated, all numerical quantities in this description indicating amounts of material and/or use are to be understood as modified by the word “about” in describing the broadest scope of the invention. Practice within the numerical limits stated is generally preferred.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

Ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

The term “and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z,” unless specifically stated otherwise, is otherwise understood with the context as used in general to convey that an item, term, etc. may be either X, Y, or Z; X and Y; X and Z; Y and Z; or X, Y, and Z (i.e., any combination of X, Y, and Z).

Throughout this application, where publications are referenced, the disclosures of these publications in their entireties are hereby referred to in this application to more fully describe the state of the art to which this invention pertains.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### EXAMPLES

### Materials and Methods

### ELISA of Gal-1, -3 and -9 in DLBCL Patient Sera

Sera were collected from 31 DLBCL patients and 31 healthy control donors from the Biospecimen Repository Facility at Baptist Health-South Florida/Miami Cancer Institute (Miami, FL) and stored at −80° C. until further analysis to assess Gal-1, -3, and -9 levels using a commercial ELISA kit (DGAL10, DGAL30, DGAL90; R&D Systems, Minneapolis, MN, USA). Absorbance was measured at 450 nm, and a standard curve was generated using a Cytation 5 reader with Gen5 software (BioTek Instruments, Winooski, VT, USA). All samples were analyzed in triplicate. Gal-1, -3, and -9 levels in DLBCL patients were compared to those of healthy control donors, with results expressed in ng/ml. Complete details of the reagents are available in Table 1.

### Gene Expression Data Collection and Processing.

The GEO browser (www.ncbi.nlm.nih.gov/geo/browse/) was searched for microarray datasets GSE2350 (comparing DLBCL (n=11) to normal memory B cells (n=5), naïve B cells (n=5) and germinal center B cells (n=17)) and GSE173263 (comparing early failure patients (n=10) to responding patients (n=29)) was analyzed using the interactive web tool GEO2R (www.ncbi.nlm.nih.gov/geo/geo2r/), which employs ‘limma’ package of R programming language for gene expression analysis (16). An adjusted p value <0.05 was considered significant. ImmuCellAI (http://bioinfo.life.hust.edu.cn/web/ImmuCellAI/) and the GSCA (Gene Set Cancer Analysis) (guolab.wchscu.cn/GSCA/#/) integrated databases were utilized to estimate the abundance of tumor-infiltrating immune cells in DLBCL samples. Statistical significance was determined using Spearman correlation with false discovery rate (FDR) correction (FDR ≤0.05).

### Cell Lines and Cell Culture.

Human B-lymphoma Raji cell line expressing luciferase (Raji-Luc⁺) purchased from the ATCC (Manassas, VA). Human B-lymphoblast-like cell line (SUDHL-6-Luc⁺) was purchased from Creative Biolabs (Shirley, NY) were cultured in RPMI-1640/10% FBS. Cell cultures were regularly validated to be mycoplasma free.

### Construction of ST6GAL1/CAR-T Cells

### Plasmid Amplification and Lentivirus Production

Anti-CD19 CAR (CD19 scFv/4-1BB/CD3) and ST6GAL1/anti-CD19 (ST6GAL1-P2A-CD19 scFv/4-1BB/CD3ζ) (transfer plasmids) were kindly provided by Dr. Avery Posey (Department of Systems Pharmacology and Translational Therapeutics, Perelman School of Medicine, University of Pennsylvania). The plasmids were amplified via heat shock transformation using STbl3 cells (Invitrogen, Carlsbad, CA, USA) and subsequently isolated with a Maxi Prep kit (QIAGEN, Hilden, Germany) according to the manufacturer's instructions. Lentiviruses were produced by transfecting HEK 293T cells (ATCC, Manassas, VA, USA) grown to 70% confluency at 37° C. in a 5% CO₂ incubator with transfer plasmids, along with the LV-MAX Lentiviral mix (Gibco™, Waltham, MA, USA) and the lentiviral packaging plasmids, pRSV.REV (Rev expression vector, Addgene #12253), pMDLg/p.RRE (Gag/Pol expression plasmid, Addgene #12251), and pVSV-G (VSV glycoprotein expression vector, Addgene #8454), combined with Opti-MEM (Thermo Fisher Scientific, Waltham, MA, USA) at a 15:18:18:7 mass-unit ratio. This DNA mixture was then added, at a 1:1 volume ratio, to a pre-warmed mixture of Opti-MEM and Lipofectamine 2000 (Thermo Fisher Scientific, Waltham, MA, USA) and incubated at room temperature for 10 minutes before being added to the HEK 293T cells in fresh culture medium. Viral supernatants were collected at 24 and 48 hours post-transfection, passed through 0.45 μm filters, and concentrated using an Amicon® Ultra Centrifugal Filter (Millipore, Burlington, MA, USA). The concentrated lentivirus was stored at −80° C., and the viral titer was determined using the Lenti-X™ Provirus Quantitation Kit (Takara Bio, Inc., Mountain View, CA, USA). Reagent details are available in Table 1.

TABLE 1
Antibodies and reagents used for ELISA, RT-qPCR, flow
cytometry, isolation/transduction/expansion of human
T cells, lentiviral production and in vivo experiments.

| Antibodies/Reagents | Source | Identifier |
|---|---|---|
| Human Galectin-1 Quantikine ELISA Kit | R&D Systems ™ | Cat# DGAL10 |
| Human Galectin-3 Quantikine ELISA Kit | R&D Systems ™ | Cat# DGAL30 |
| Human Galectin-9 Quantikine ELISA Kit | R&D Systems ™ | Cat# DGAL90 |
| Human ST6GAL1 TaqMan ® primer | Thermo Scientific | AssayID: Hs00949382_m1 |
| Human ST3GAL1 TaqMan ® primer | Thermo Scientific | AssayID: Hs00161688 |
| Human B4GALT1 TaqMan ® primer | Thermo Scientific | AssayID: Hs00155245_m1 |
| Human MGAT4 TaqMan ® primer | Thermo Scientific | AssayID: Hs00608465 |
| Human MGAT5 TaqMan ® primer | Thermo Scientific | AssayID: Hs00159136_m1 |
| Human GCNT1 TaqMan ® primer | Thermo Scientific | AssayID: Hs01922706_s1 |
| Human GCNT2 TaqMan ® primer | Thermo Scientific | AssayID: Hs00377334_m1 |
| Human 18s TaqMan ® primer | Thermo Scientific | AssayID: Hs03003631_g1 |
| Applied Biosystems ™ TaqMan ™ Fast | Thermo Scientific | Cat# 4444556 |
| Advanced Master Mix | | |
| TaqMan ® Fast Advanced Master Mix | Thermo Scientific | Cat# 4444557 |
| RNeasy Plus Mini Kit (250) | QIAGEN | Cat# 74136 |
| SuperScript ™ VILO ™ cDNA Synthesis | Invitrogen ™ | Cat# 11-754-050 |
| Kit | | |
| Biotin-SP (long spacer) AffinityPure Goat | Jackson | Cat # 115-065-072 |
| Anti-Mouse IgG, F(ab′)₂ fragment specific | ImmunoResearch | |
| | Laboratories | |
| Alexa Fluor ® 647 Rat IgG2a, κ Isotype Ctrl | Biolegend | Cat# 400526 |
| Antibody | | |
| LIVE/DEAD ™ Fixable Aqua Dead Cell | Fisher | Cat# 50-112-1525 |
| Stain Kit, for 600 nm excitation | | |
| PE anti-human CD3 Antibody | Biolegend | Cat# 300408 |
| FITC anti-human CD4 Antibody | Biolegend | Cat# 317408 |
| PerCP anti-human CD8a Antibody | Biolegend | Cat# 300922 |
| Dead Cell Apoptosis Kit with Annexin V | Fisher | Cat# V13241 |
| Alexa Fluor ™ 488 & Propidium Iodide (PI) | | |
| PE Streptavidin (High Concentration) | Fisher | Cat # 405245 |
| Zombie Aqua ™ Fixable Viability Kit | Biolegend | Cat # 423101 |
| Alexa Fluor ® 647 anti-mouse/human Mac-2 | Biolegend | Cat# 125408 |
| (Galectin-3) Antibody | | |
| APC anti-human Galectin-9 Antibody | Biolegend | Cat# 348908 |
| Anti-human Galectin-1 antibody | Abcam | Cat# ab25138 |
| APC Mouse IgG1, κ Isotype Ctrl | Biolegend | Cat# 400120 |
| Antibody | | |
| Biotin Rat IgG2a, κ Isotype Ctrl | Biolegend | Cat# 400504 |
| Antibody | | |
| Brilliant Violet 421 ™ Rat IgG2b, κ | Biolegend | Cat# 400640 |
| Isotype Ctrl Antibody | | |
| FITC Mouse IgG1, k Isotype Ctrl (FC) | Biolegend | Cat# 981802 |
| PerCP Mouse IgG1, k Isotype Ctrl | Biolegend | Cat# 400148 |
| PE Mouse IgG2a, k Isotype Ctrl (FC) | Biolegend | Cat# 400212 |
| APC anti-mouse/human IL-5 | Biolegend | Cat# 504306 |
| Paraformaldehyde Solution | Thermo Scientific | Cat# NC1660551 |
| Solanum Tuberosum (Potato) Lectin (STL, | Vector | Cat# L-1160-5 |
| PL), Unconjugated | Laboratories | |
| Maackia Amurensis Lectin II (MAL II), | Vector | Cat# B-1265 |
| Biotinylated | Laboratories | |
| Sambucus Nigra Lectin (SNA), Fluorescein | Vector | Cat# FL-13012 |
| | Laboratories | |
| Recombinant human galectin-1 (rhGal1) | R&D Systems ™ | Cat# 1152-GA-050 |
| Recombinant human galectin-3 (rhGal3) | R&D Systems ™ | Cat# 1154-GA-050 |
| Recombinant human galectin-9 (rhGal9) | R&D Systems ™ | Cat# 2045GA050 |
| Recombinant Human IL-5 Protein | R&D Systems ™ | Cat# 205-IL-005/CF |
| Human IL-2 Recombinant Protein | R&D Systems ™ | Cat# 202-IL-050/CF |
| ImmunoCult ™ Human CD3/CD28 T Cell | Stem Cell | Cat# 10971 |
| Activator | Technologies | |
| Naive CD4+ T Cell Isolation Kit II, human | Miltenyi Biotec | Cat# 130-094-131 |
| Naive CD8+ T Cell Isolation Kit, human | Miltenyi Biotec | Cat# 130-093-244 |
| Pan T Cell Isolation Kit, human | Miltenyi Biotec | Cat # 130-096-535 |
| HiPure Plasmid Filter Maxiprep Kit | Invitrogen ™ | Cat# K210017 |
| ViraPower ™ Lentiviral Gateway ™ | Invitrogen ™ | Cat# K496000 |
| Expression Kit | | |
| One Shot Stbl3 E. coli | Invitrogen ™ | Cat# C7373-03 |
| Lipofectamine 2000 | Invitrogen ™ | Cat# 11668027 |
| Bright-Glo ™ Luciferase Assay System | Promega | Cat# E2610 |
| VivoGlo ™ Luciferin, In Vivo Grade | Promega | Cat# P1042 |
| Firefly Luciferase Lentivirus (Puromycin) | BPS Bioscience | Cat# 79902-P |
| Expression Negative Control Lentivirus | BPS Bioscience | Cat# 79692-P |
| (Puromycin) | | |
| Nano-Glo ® Luciferase Assay System | Promega | Cat# N1110 |
| Nano-Glo ® Fluorofurimazine In Vivo | Promega | Cat# N4110 |
| Substrate | | |

### Isolation, Transduction, and Expansion of Primary Human T Cells Expressing CAR or ST6GAL1 and CAR

Peripheral blood mononuclear cells (PBMCs) were isolated from normal healthy donor leukopacks (OneBlood, Inc., Miami, FL, USA) using Ficoll-Paque (GE Healthcare, Chicago, IL, USA) via gradient centrifugation and frozen down and stored at LN₂. PBMC aliquots were thawed, washed, and subjected to immunomagnetic cell separation using the Pan T Cell Isolation Kit (Miltenyi Biotec, Auburn, CA, USA). T cells were stimulated with ImmunoCult™ Human CD3/CD28 T Cell Activator (Stem Cell Technologies, Vancouver, BC, Canada; 25 μl/ml) and transduced one day after activation with lentiviral vectors at a multiplicity of infection (MOI) of 3. To support T cell expansion, human IL-2 (R&D Systems, Minneapolis, MN, USA) was added every other day for 10 days at a final concentration of 100 IU/ml. Day-10 expanded T cells refer to CD3/CD28-activated, IL-2-expanded T cells cultured for 10 days in parallel with CAR-T manufacturing but not subjected to lentiviral transduction. Reagent details are available in Table 1.

### Validation of CAR and ST6GAL1 Expression in Transduced Primary Human T Cells

To confirm expression of CAR and ST6GAL1 expression in 10-day expanded transduced primary human T cells, cells were analyzed by flow cytometry using moAbs raised against murine Ig domains in CAR and Sambucus nigra Agglutinin (SNA) (0.5 μg/ml), which recognizes α2,6 sialylated structures synthesized by ST6GAL1. Cells were washed with phosphate-buffered saline (PBS) containing 1% BSA and stained on ice. Singlets were identified using FSC-H vs. FSC-A and SSC-H vs. SSC-A, followed by gating based on forward and side scatter characteristics. All staining included a live/dead cell marker (Zombie, BioLegend, San Diego, CA, USA) and surface detection of CD19+ CAR using a biotin-conjugated goat anti-mouse F(ab′)₂ antibody (Jackson ImmunoResearch, West Grove, PA, USA) followed by PE-conjugated streptavidin (BioLegend, San Diego, CA, USA). For detecting α2-6-linked sialic acids, SNA (Vector Laboratories, Burlingame, CA, USA) was used. Data are presented as mean fluorescence intensity (MFI±SEM). Stained cells were immediately acquired using BD FACS Diva 6.1 software and analyzed with FlowJo (BD Biosciences, San Jose, CA, USA), with electronic gating for lymphocytes and doublet discrimination.

### RT-qPCR Analysis.

RT-qPCR analysis was conducted on RNA extracted from naïve T cells (control) and T cells activated for 2 days (Day 2), 5-days (Day 5) or for 10-days (Day 10), and CAR-T cell formulations. cDNA was synthesized, and real-time quantitative RT-PCR was performed to assess genes related to galectin ligand-building/suppressing ligand synthesis, ST6GAL1, B4GALT1, B3GNT2, GCNT1, GCNT2, MGAT5 and ST3GAL1. Data was normalized to housekeeping gene 18S, and relative transcript levels were analyzed using the 2^{(-ΔΔCt)} method from 3 independent experiments. Reagent details are available in Table 1.

### Flow Cytometry.

Flow cytometry assays incorporated cell staining reagents: live/dead cell marker Zombie, 7-AAD (Biolegend). For assessment of endogenous Gal-3 expression on target cells, cells were stained with an anti-Gal-3 antibody (BioLegend) together with an isotype-matched control antibody to define background signal. For recombinant galectin binding, recombinant human (rh) Gal-1, -3 and -9 (All from R&D Systems), anti-Gal-1, -Gal-3, and -Gal-9 Abs (Biolegend) as well as Abs to differentiation markers, CD3, CD4 and CD8 were used. Cells were stained with rhGal-1, -3 and -9 at 3 μg/ml, 5 μg/ml and 1 μg/ml, respectively, with or without 50 mM lactose to confirm galactose-binding dependency, and then stained with respective anti-Gal Ab or isotype-matched control Ab. Plant lectin staining was performed using Sambucus nigra agglutinin (SNA; Vector Laboratories) to detect α2,6-linked sialylation, Maackia amurensis lectin II (MAL II; Vector Laboratories) to detect α2,3-linked sialylation, and Solanum tuberosum lectin (STL; Vector Laboratories) to detect poly-LacNAc residues. Cells were washed with PBS containing 1% BSA and stained on ice with the respective biotinylated lectins, along with a live/dead marker (Zombie; Biolegend) followed by FITC (Biolegend). CAR detection was performed using biotin-conjugated goat anti-mouse F(ab′)₂ antibody (Jackson ImmunoResearch) followed by PE-streptavidin (Biolegend). Singlets were gated using FSC-H vs. FSC-A and SSC-H vs. SSC-A, and lymphocytes were identified based on forward/side scatter. Data were reported as mean fluorescence intensity (MFI±SEM). Stained cells were immediately acquired using BD FACS Diva 6.1 software and analyzed in FlowJo (BD Biosciences). Background fluorescence were controlled using appropriate negative controls depending on the assay. For galectin binding experiments, samples stained in the presence of 50 mM lactose were used as competitive binding controls, and mean fluorescence intensities (MFI) from lactose-treated samples were subtracted from experimental values. For antibody-based staining, isotype-matched control antibodies and/or unstained controls were used to define background signal, and corresponding MFIs were subtracted prior to data analysis. Reagent details are provided in Table 1. All experiments were performed 3-times to obtain statistical significance.

### Cell Proliferation Assay.

Cell proliferation was evaluated using the Cell Counting Kit-8 (CCK-8; Dojindo, Japan) following the manufacturer's protocol. Cells were seeded in 96-well plates at a density of 2,000 cells per well. Proliferation was assessed at 0, 24, 48, and 72 hours by adding 10 μL of CCK-8 reagent to each well and incubating at 37° C. for 3 hours. Absorbance was measured at 450 nm using a Cytation 5 microplate reader (BioTek, Winooski, VT, USA). All experiments were performed in 3-times to ensure statistical significance.

### MALDI-TOF MS N-Glycomic Analysis of Human T Cells.

CAR-T cells and control T cell cohorts were treated as described by Sweeney et al. (Sweeney J G, Liang J, Antonopoulos A, Giovannone N, Kang S, Mondala T S, et al. Loss of GCNT2/I-branched glycans enhances melanoma growth and survival. Nat Commun. 2018; 9(1):3368). Cell pellets were sonicated in ice-cold lysis buffer (25 mM Tris, 150 mM NaCl, 5 mM EDTA, and 1% CHAPS, pH 7.4), dialyzed in Slide-A-Lyze™ dialysis cassettes (3.5 kDa cutoff; Thermo), reduced by DTT in 4 M guanidine-HCl (Thermo), carboxymethylated by IAA, and then digested by trypsin (Sigma, 24 h, 37° C.). The digested samples were purified using Oasis plus short HLB Sep-Pak (Waters) extraction cartridges. N-glycans were released by PNGase F (recombinant from Escherichia coli, E.C. 3.5.1.52; Roche) digestion (24 h, 37° C.) and purified by classic short C18-Sep-Pak (Waters). Released N-glycans were permethylated with methyl iodide and then purified by classic short C18-Sep-Pak (Waters) prior to MS analysis. All permethylated glycans were dissolved in 10 μl methanol. One 1 μl of sample was mixed with 1 μl of matrix (10 mg/ml 3,4-diaminobenzophenone (Acros Organics) in 75% (v/v) aqueous acetonitrile) and spotted onto a MALDI plate. MS data were acquired using a 4800 MALDI-TOF (Applied Biosystems, Darmstadt, Germany) mass spectrometer. The 4700 calibration standard kit, Calmix (Applied Biosystems) was used as the external calibrant for MS.

Data were analyzed using Data Explorer™ (version 4.9, Applied Biosystems) and GlycoWorkBench. The glycomic data were annotated based on monosaccharide composition derived from the molecular ion m/z value, knowledge of glycan biosynthetic pathways, the isotopic peak cluster patterns, and MS derived fragmentation.

### Cell Death Assays.

To investigate the effect of Gal-3 on apoptotic induction, T cells (1×10⁵/mL) were cultured in 96-well plates with rhGal-3 (R&D systems) (4 μg/mL±50 mM lactose) for 16 h. Cells were stained with FITC-annexin-V and PI, then analyzed by flow cytometry to determine viable (annexin-V⁻ PI⁻), early apoptotic (annexin-V⁺ PI⁻), and late apoptotic/necrotic (annexin-V⁺ PI⁺) populations. All experiments were performed 3-times to obtain statistical significance. Reagent details are available in Table 1.

### Cytokine Profiling Arrays.

Cells were treated with rhGal-3 (4 μg/ml) with/without 50 mM lactose for 16 h. Supernatants were collected and pro-/anti-inflammatory cytokines and chemokines, C5a, CD40L, G-CSF, GM-CSF, CXCL1, IL-8, IL-16, IL-18, CCL5, IL-1ra, SerpinE1, IL-1α, IL-1β, IL-2, IL-4, IL-5, IL-6, IL-10, IL-12p70, IL-13, IL-17, IL-17E, IL-23, IL-27, IL-32α, MIF, ICAM-1, IFN-γ, CXCL10, CXCL11, CCL2, CCL3, CCL4, CXCL12, TNF-α and TREM-1, were measured using a Proteome Profiler Human Cytokine Array Kit (R&D systems), according to the manufacturer's instructions. Streptavidin-HRP detection agents was subsequently used to develop the blot with the intensity of the spot in direct proportion to the amount of cytokine bound. Membranes were visualized with the iBright™ FL1500 Imaging System (Invitrogen) and analyzed using iBright™ Analysis Software (Invitrogen). All experiments were performed 3-times to obtain statistical significance. Reagent details are available in Table 1.

### Intracellular IL-5 Analysis.

To investigate the effect of Gal-3 on cytokine induction, cells were incubated with 4 μg/ml rhGal-3 (R&D systems) with 200×10³/ml CAR-T cells, ST6^{OE} CAR-T cells, and Day 10-expanded T cells (control) in complete medium in 96-well plates for 16 h in 200 μL at 37° C. For 4-6 h, cells were activated with a pre-mixed cocktail containing PMA, ionomycin, and Brefeldin A (BD Biosciences) to block cytokine secretion. Cells were then washed twice with PBS buffer and stained with Zombie Aqua™ Fixable Viability (Biolegend) for 20 min at room temperature. Cells were then washed in cell staining buffer (BD Biosciences) following immunostaining with a surface stain cocktail containing the following antibodies: CD8a PerCP/Cyanine5.5 (Biolegend), CD4 BV605 (Biolegend), and CD3ε APC/Fire 750 (Biolegend) in Stain Buffer (BD Biosciences). After 30 min of incubation, cells were washed twice and incubated with 500 μL of BD Fixation and Permeabilization Solution (BD Biosciences) at 4° C. for 20 min, washed twice with BD Perm/Wash Buffer (BD Biosciences), and stained with a cocktail of fluorescently labeled anti-IL-5 APC (Biolegend) in Stain Buffer (BD Biosciences). After 30 min, the cells were washed once with 1× Perm/Wash Buffer (BD Biosciences) and resuspended in 200 μL of Stain Buffer (BD) prior to flow cytometric analysis. All experiments were performed 3-times. Reagent details are available in Table 1.

### Tumoricidal Assay.

To evaluate tumoricidal activity and the effect of IL-5 in tumoricidal activity, T cells were all incubated+/−4 μg/ml rhGal-3, +/−1 or 10 ng of rhIL-5 in co-cultures of human CD19⁺ Raji-luc⁺ or SUDHL-6-luc⁺ target cells in 96-well plates (VWR) RPMI-1640/10% FBS. Effector:Target (T cell:tumor cell-luc⁺) at 20:1 for a 16 h incubation in a total volume of 100 μL RPMI-1640/10% HI-FBS. Post-incubation, T cell and tumor cell-luc⁺ co-cultures were lysed using Bright-GLO reagent (Promega) and/or Nano-Glo Live Cell Assay System (Promega) according to the manufacturer's instruction. Luminescence was analyzed using AMI HT imaging system. Gal-3^{high} and Gal-3^{low} target cell designations were defined based on relative endogenous Gal-3 expression assessed by flow cytometry under standard culture conditions prior to tumoricidal assays. SUDHL-6-luc⁺ cells consistently exhibited higher Gal-3 expression compared with Raji-luc⁺ cells across independent experiments and were designated Gal-3^{high}, whereas Raji-luc⁺ cells were designated Gal-3^{low}. All experiments were performed 3-times to obtain statistical significance. Reagent details are available in Table 1.

### In Vivo Anti-Tumor Assays.

To compare anti-tumor efficacy of ST6^{OE} CAR-T cells with control CAR-T cells, equal numbers of 6-8-week-old male/female NOD-SCID IL-2Rγ-deficient (NSG) mice were inoculated intravenously (i.v.) with 10⁵ Raji-luc⁺ cells (100 μl media). After 7 days, media or T cell cohorts (5×10⁶ in 100 μl media) were i.v. transferred. Tumor burdens were assayed weekly for 35 days by i.p. administration of 100 μl of 15 mg/ml D-luciferin solution (VivoGlo™ Luciferin) (Promega) and bioluminescent imaging (BLI) with AMI HT imager. For BLI, mice were anesthetized with 2% isoflurane and maintained under anesthesia by continuous inhalation of isoflurane until imaging was complete. Ventral and lateral images of the mice were taken 10 min later using AMI HT imaging system and quantified using the Aura Imaging Software. Tumor growth and survival were analyzed using growth rates and Kaplan-Meier curves. All experimental procedures were conducted as per Florida International University Institutional Animal Care and Use Committee (IACUC) guidelines and IACUC protocol #IACUC-25-049-AM02.

### Statistical Analysis.

Prism 8.0 software (GraphPad) was used for statistical analysis Data distribution was assessed using the Shapiro-Wilk normality test. For comparisons between two groups with non-normally distributed data, the Mann-Whitney U test was used and Student's t-test was applied when normality assumptions were met. For comparisons involving more than two groups, the Kruskal-Wallis test followed by Dunn's multiple-comparisons correction was applied. For experiments involving two independent variables and repeated measurements, data were analyzed using a two-way repeated-measures ANOVA with Sidak's multiple-comparisons test.

To account for potential age-related confounding, age-adjusted analyses were performed using multiple linear regression, with disease status (healthy vs DLBCL) and age included as independent variables. Regression analyses were conducted in Prism using one observation per individual. For survival analysis, Gehan-Breslow-Wilcoxon test was used. Throughout, data are presented as the means SEM, unless otherwise noted. A p value of p<0.05 was considered statistically significant.

For transcriptomic datasets and immune-infiltration analyses (ImmuCellAI, GSCA), multiple-testing correction was performed using Spearman's rank correlation, with statistical significance determined following false discovery rate (FDR) correction using the Benjamini-Hochberg method (FDR ≤0.05).

### Example 1—CAR-T Cells Show Remarkable Binding to Gal-1 and Gal-3

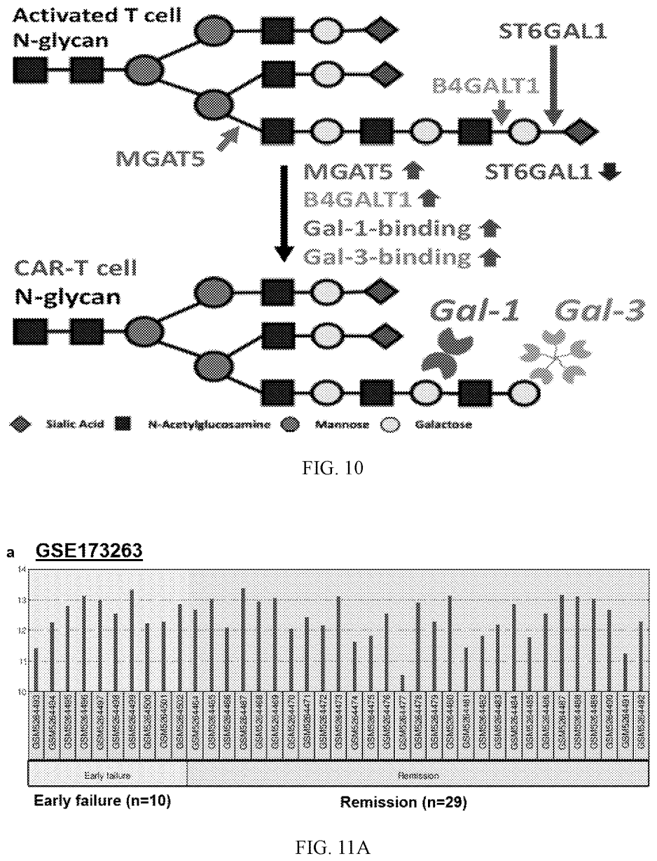

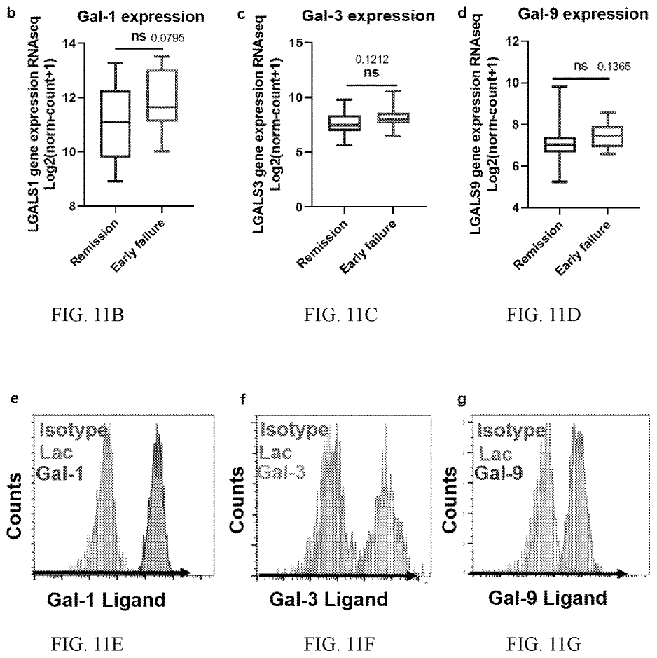

To assess the Gal-1, -3 and -9-binding activity on CAR-T cells, anti-CD19 CAR-T cells (anti-CD19 scFv/4-1BB/CD3ζ) were generated with 4-1BB and CD3zeta co-stimulatory domains (CD19/BBz) from human naïve T cell MACS-sorted from PBMC (n=6) (naive Pan T Cell Isolation Kit; Miltenyi), activated for 2-days with anti-CD3/CD28 beads (ImmunoCult™ CD3/CD28 T cell activator; Stem Cell Tech.), transduced with lentivirus containing CD19 CAR, expanded for 10-days, and assayed for binding activity by flow cytometry using optimized concentrations of rhGal-1 (10 μg/ml), rhGal-3 (5 μg/ml) or rhGal-9 (4 μg/ml) and corresponding anti-Gal Ab, as well as anti-CD4/CD8 moAbs to discern levels among T cell subsets. Negative controls included staining with isotype-matched Abs in place of anti-Gal Abs or staining with rhGal and anti-Gal Abs in the presence of lactose, a competitive inhibitor of galectin binding (FIGS. 11E-11G).

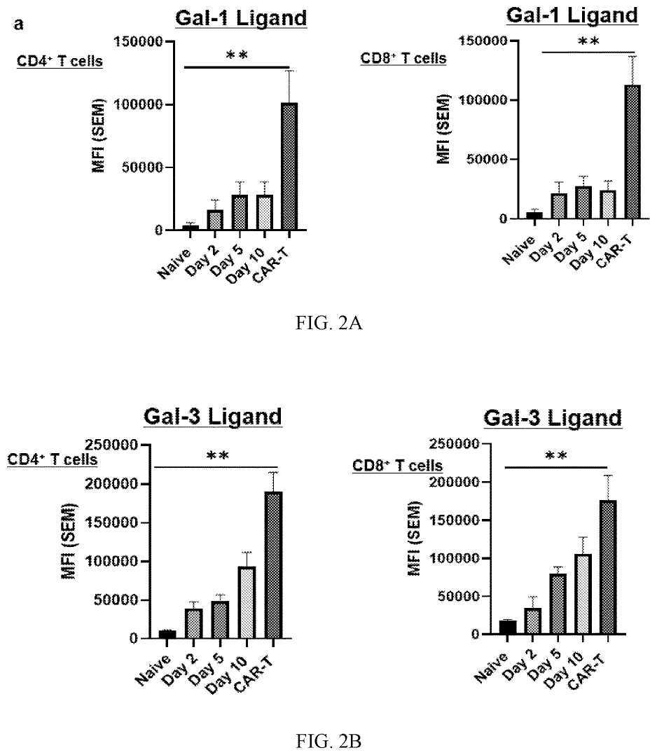

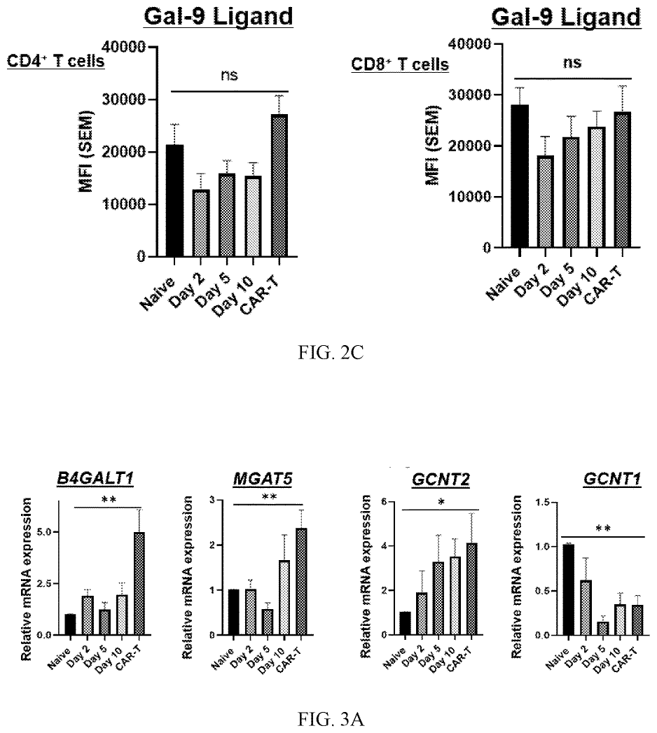

Compared with naïve, Day 2, Day 5 or Day 10 T cells, both CD4⁺ and CD8⁺ CAR-T cells exhibited significantly more Gal-1- (FIG. 2A) (p<0.001) and Gal-3-binding (FIG. 2B) (p<0.01). Adding control 10 mM lactose to rhGal-1, -3, or -9 neutralized galectin-binding and Gal-9 binding to CAR-T cells (FIG. 2C) was not significantly different than binding to controls, indicating selective enhancement of Gal-1 and Gal-3 ligand availability following CAR-T cell manufacturing.

Based on elevated Gal-1 and -3 binding to CAR-T cells compared with binding to Day 10-expanded T cells, it is apparent that transduction of CAR-expressing lentivirus, independent of CAR scFv-dependent CD19 Ag recognition and cell activation can boost Gal-1 and Gal-3 glycan ligand-building enzymes.

### Example 2—Inhibitor of Gal-1/-3 Binding Activity, α2,6 Sialyltransferase 1 (ST6GAL1), is Depressed, Whereas Gal-1/-3 Glycan Ligand-Building Enzymes, β1,6 N-Acetylglucosaminyltransferase 5 (MGAT5) and β1,4 Galactosyltransferase 1 (B4GALT1) are Elevated in CAR-T Cells

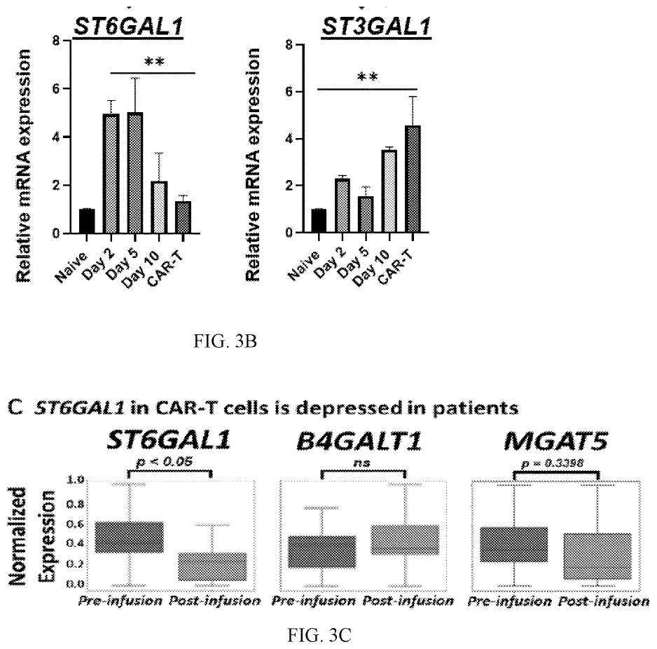

CAR-T cells (CD19/BBz) were generated from human naïve T cell MACS-sorted PBMC (n=6 leukopacks purchased from OneBlood, Inc) as noted above, and their galectin-synthesizing (MGAT5, GCNT1 and B4GALT1) and -inhibitory (ST6GAL1 and ST3GAL1) glycosyltransferases were compared with Naïve, Day 2-activated, Day 5-expanded T cells, and Day 10-expanded non-transduced T cells by RT-qPCR analysis. After purifying RNA and conversion to mRNA and cDNA from each cell group (RNAEasy Kit, Qiagen), RT-qPCR analysis was performed. After GAPDH normalization and compared with expression in Naïve cells, β1,6 N-acetylglucosaminyltransferase V (MGAT5), β1,6 N-acetylglucosaminyltransferase 2 (GCNT2) and β1,4 galactosyltransferase 1 (B4GALT1) were significantly higher (p<0.01, p<0.05 and p<0.01) (FIG. 3A). These enzymes promote increased N-glycan branching and antennae extension with N-acetyllactosamine (LacNAc) units, which confer high affinity galectin binding. In contrast, expression of core 2 β1,6 N-acetylglucosaminyltransferase 1 (GCNT1) was unexpectedly significantly lower (p<0.01) (FIG. 3A). Further, galectin-binding inhibitor, β-galactoside α2,6 sialyltransferase 1 (ST6GAL1) was significantly depressed in CAR-T cells (p<0.01) (FIG. 3B). ST6GAL1 action caps terminal β-galactose residues with α2,6-linked sialic acid to block Gal-1 and Gal-3 ligand recognition, while GCNT1 generates core 2 O-glycans that can be elongated into galectin-binding LacNAc structures. Interestingly, expression of β-galactoside α2,3 sialyltransferase 1 (ST3GAL1) (FIG. 3B) (p<0.01) was increased in CAR-T cells; so, together with GCNT1 loss, this expression pattern likely resulted in α2,3 sialylated core 1 O-glycans that are poor galectin ligands.

Together, these results indicated that the Gal-1 and Gal-3 ligand-generating glycome of CAR-T cells was represented by a GCNT1^{low}/ST3GAL1^{high} O-glycan program and an N-glycan program driven by upregulated expression of MGAT5/B4GALT1/GCNT2 branching/elongation enzymes combined with downregulated expression of ST6GAL, consistent with increased Gal-1 and Gal-3 ligand availability (FIG. 3D).

To corroborate these data, levels of MGAT5, B4GALT1, and ST6GAL1 in CD19 CAR-T cells pre- and post-infusion into DLBCL patients were analyzed. Transcriptomics of individual CAR-T cells collected from patients that were sorted, counted, processed for 5′ single-cell RNA-sequencing with feature barcoding and TCR clonotype analysis on the 10× Genomics platform, and sequenced by HighSeq 4000. Pre-infusion expression values from GSE151511 were compared with values from post-infusion expression from GSE168940. Gene expression data from GSE151511 and GSE168940 were normalized using min-max normalization and displayed as boxplots normalization. Compared with expression in pre-infused CAR-T cells, ST6GAL1 in post-infused CAR-T cells was lower (p<0.05), while B4GALT1 and MGAT5 levels were insignificant (FIG. 3C). These data suggested that transduced CAR-T cells are vulnerable to galectin-binding, potentially through significant lowering of galectin-binding inhibitory enzyme, ST6GAL1.

Taken together, these studies suggested that robust Gal-1 and Gal-3 binding to CD19/BBz cells may be most impacted by lowering ST6GAL1 and that restoring ST6GAL1 levels in CAR-T cells can obviate Gal-1 and Gal-3 immunomodulatory effects.

### Example 3—Anti-CD19 CAR-T Cell N-Glycome was Represented by High N-Glycan Branching/Elongation with Concomitant Reduction in α2,6 Sialylation

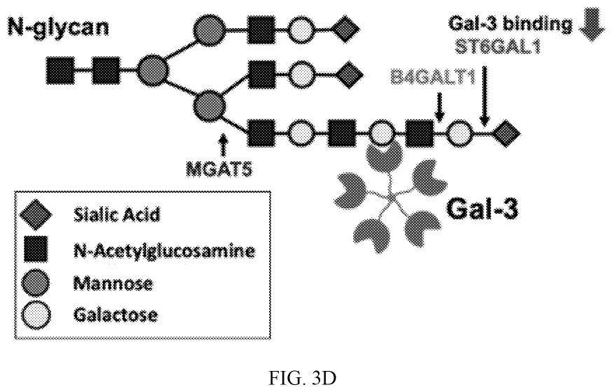

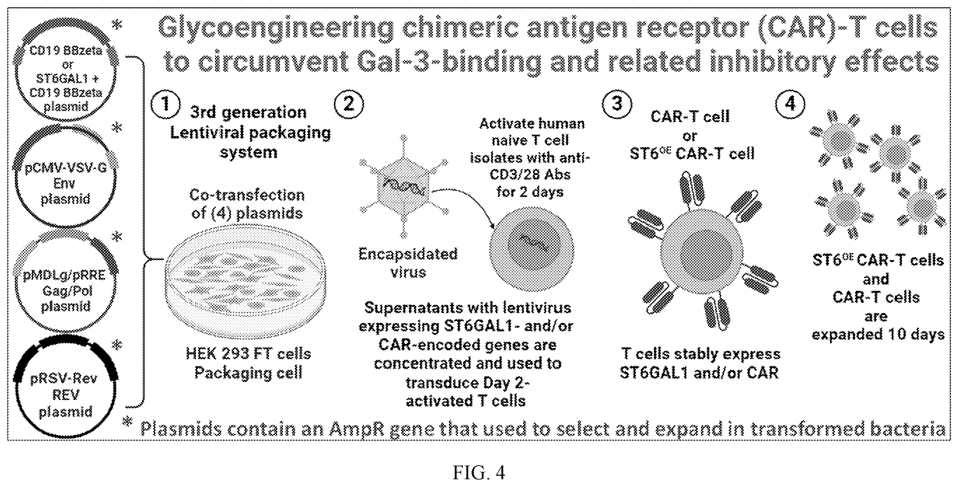

As an approach to enforce α2,6 sialylation and blunt Gal-1 and Gal-3 binding activity, human T cells were transduced with α2,6 sialyltransferase 1, ST6GAL1. Accordingly, Day 2-activated T cells were transduced with lentivirus containing CD19/BBz or ST6GAL1/CD19/BBz to generate CAR-T cells or ST6^{OE} CAR-T cells (FIG. 4).

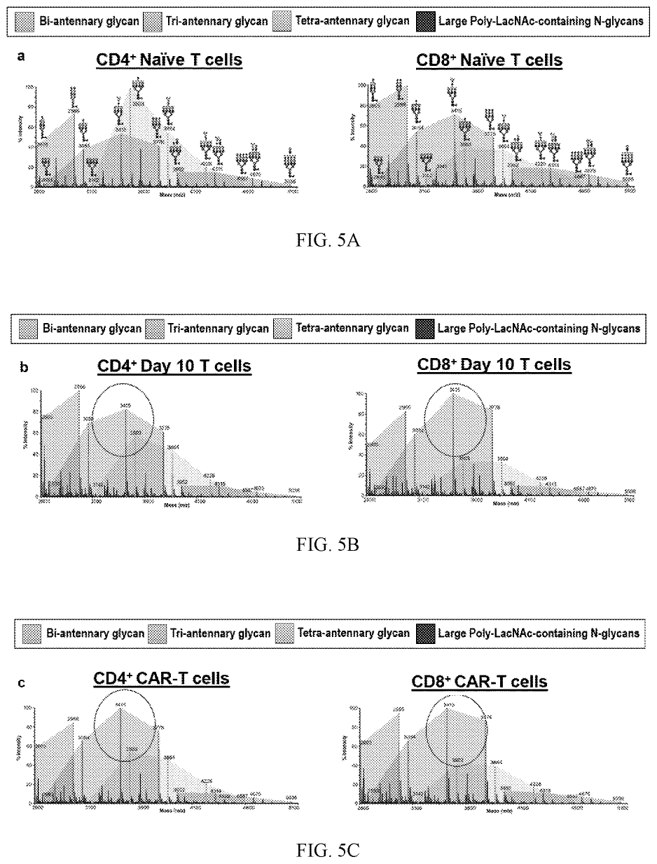

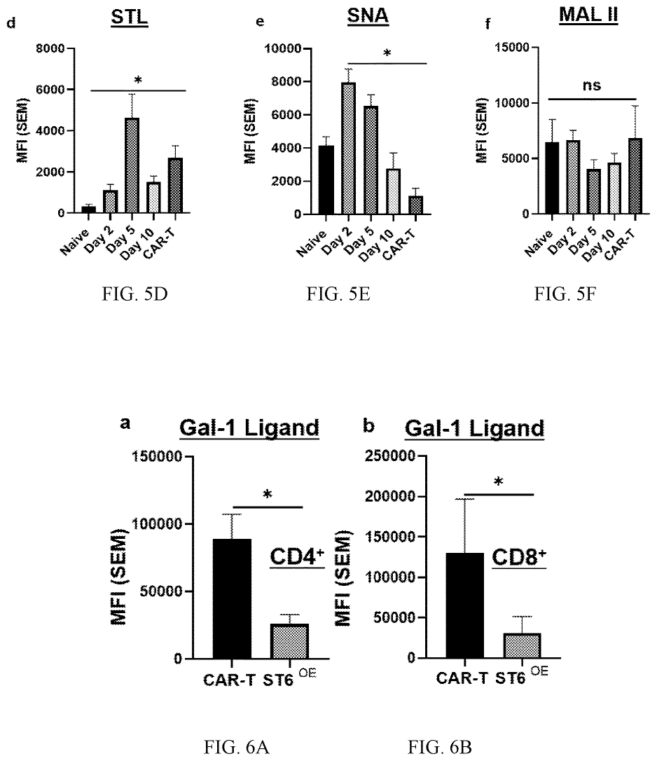

Profiling of cell N-glycans by Matrix-Assisted Laser Desorption/Ionization-Time of Flight mass spectrometry (MALDI-TOF MS) revealed an enrichment of highly branched N-glycan structures in anti-CD19 CAR-T cells. As shown in the mass spectra (FIG. 5A), naïve T cells predominantly displayed smaller, bi-antennary complex N-glycans (green-shaded peaks); whereas Day 10 non-transduced expanded T cells (FIG. 5B) and CAR-T cells (FIG. 5C) displayed greater peaks corresponding to tri-antennary N-glycans (blue-shaded peaks). Tri-antennary N-glycans contain poly-N-acetyllactosamine (poly-LacNAc) chains that serve as glycan ligands for Gal-1 and Gal-3, providing a structural basis for elevated Gal-1 and Gal-3 binding capacity.

Cell surface staining with plant lectins corroborated MS N-glycomics structural findings, showing that, compared with naïve T cells, anti-CD19 CAR T cells significantly bound Solanum tuberosum (potato) lectin (STL), which specifically recognizes poly-LacNAc motifs (p<0.05) (FIG. 5D). Conversely, binding of Sambucus nigra agglutinin (SNA), which recognizes α2,6 sialylated LacNAc on N-glycans, was markedly reduced on CD19 CAR-T cells (p<0.05) (FIG. 5E). This loss of SNA reactivity was consistent with the downregulation of ST6GAL1 and fewer Gal-1 and Gal-3 ligand inhibitory α2,6-sialylated LacNAc caps on CAR-T cell N-glycan antennae. Additionally, Maackia amurensis lectin II (MAL II), which binds α2,3 sialylated LacNAc, showed no significant difference between CAR-T cells and naïve T cells, suggesting that overall α2,3 sialylation levels were largely maintained on CAR-T cells (FIG. 5F). These lectin-binding results together with MALDI-TOF MS data illustrated that, relative to naïve and non-transduced expanded T cell controls, CAR-T cells displayed a signature glycome enriched for poly-LacNAc structures on multi-antennary N-glycans with diminished α2,6 sialylation.

### Example 4—Elevated Gal-3 Levels are Associated with DLBCL Patient Serum, Tumor-Intrinsic Expression, and Macrophage-Rich TME

To assess whether immunosuppressive galectins are enriched in DLBCL-associated disease settings, serum levels of Gal-1, -3, and -9 in 31 DLBCL patients and 31 age-matched healthy controls were analyzed (Table 2).

TABLE 2
Demographics and clinical features of patients
with DLBCL and healthy controls

| | |
|---|---|
| Patients with | Healthy |
| DLBCL | Controls |
| (n = 31) | (n = 31) |

Sex, n (%)

| | | | | |
|---|---|---|---|---|
| Male | 13 | (41.9) | 12 | (38.7) |
| Female | 18 | (58.1) | 19 | (61.3) |
| Race, n (%) | | | | |
| African American | 3 | (9.7) | 3 | (9.7) |
| White | 3 | (9.7) | 12 | (38.7) |
| White Hispanic | 25 | (80.6) | 16 | (51.6) |

| | | |
|---|---|---|
| Age at diagnosis, | 63.5 ± 16.5 | 54.3 ± 13.2 |
| y (mean ± SD) | | |

| | | |
|---|---|---|
| DLBCL stage, n (%) | | |
| Primary DLBCL | 10 | (32.3) |
| Stage I | 1 | (3.2) |
| Stage II | 9 | (29.1) |
| Metastatic DLBCL | 21 | (67.7) |
| Stage III | 3 | (9.6) |
| Stage IV | 18 | (58.1) |
| Treatment at Biopsy, n (%) | | |
| CAR-T Cell therapy | 1 | (3.2) |
| Chemotherapy | 26 | (83.9) |
| Chemotherapy and | 4 | (12.9) |
| Radiation | | |

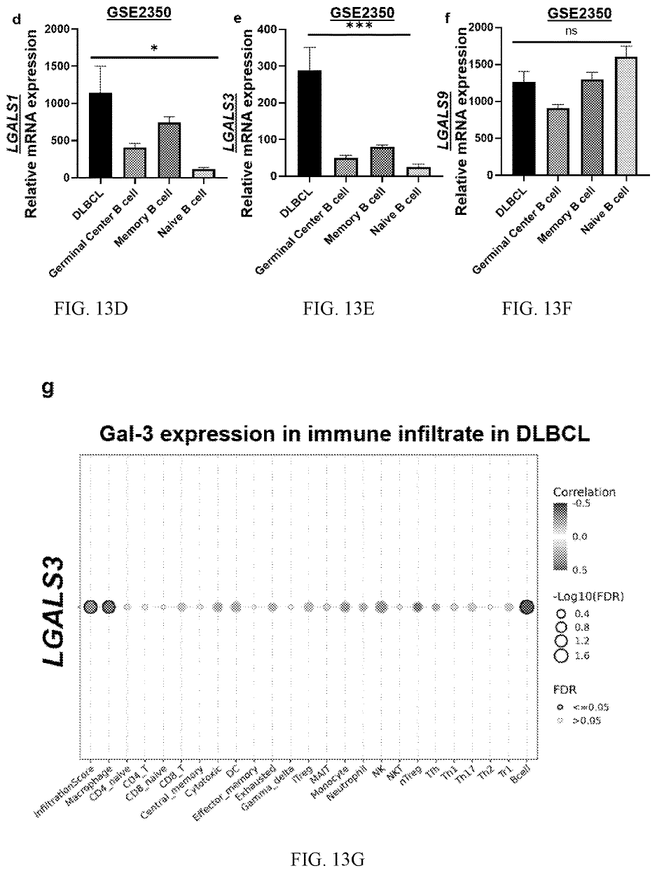

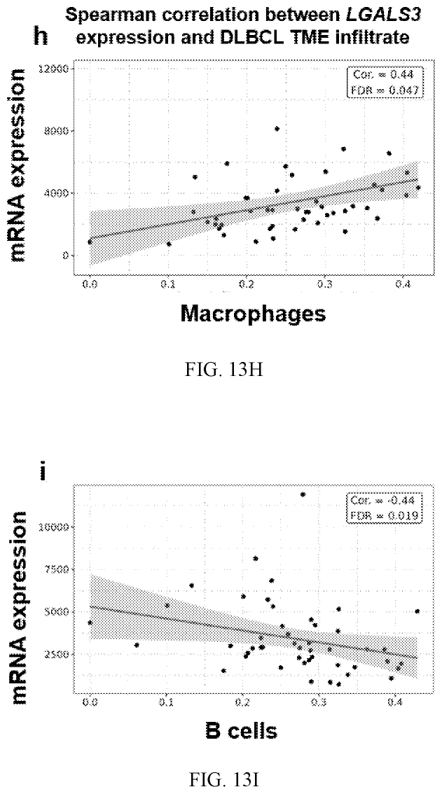

Serum Gal-1 levels showed no significant difference between groups (FIG. 13A). In contrast, DLBCL patients exhibited significantly elevated levels of Gal-3 (7.17±0.50 ng/mL vs. 9.07±0.50 ng/mL, p<0.01) (FIG. 13B) and Gal-9 (5.468±0.53 ng/mL vs. 7.183±0.53 ng/mL, p<0.01) (FIG. 13C) compared to healthy individuals after adjusting for age using multiple linear regression. Age was also found as an independent predictor of Gal-3 levels (p<0.001). These findings indicate that circulating Gal-3 and Gal-9 are elevated in DLBCL-associated disease states.

To evaluate tumor-associated galectin expression, the microarray dataset GSE2350 generated on Affymetrix U95 (GL91) platform was then analyzed and LGALS1, LGALS3, and LGALS9 transcription were compared in DLBCL samples (n=11) with normal germinal center B cells (n=17), memory B cells (n=5), and naïve B cells (n=5) (FIGS. 13D-13F). Within this context, Gal-1 (p<0.05) and Gal-3 (p<0.001) were significantly upregulated in DLBCL relative to normal mature B cell subsets (FIGS. 13D and 13E), indicating altered galectin expression within lymphoma tissues. Next, the RNA-seq dataset GSE173263 was interrogated to compare galectin expression between DLBCL patients with early treatment failure (primary refractoriness or relapse within 12 months; n=10) and patients achieving remission (n=29) (FIG. 11A). Although Gal-1, Gal-3, and Gal-9 transcripts trended higher in early-failure patients, these differences did not reach statistical significance (FIGS. 11B-11D), suggesting that galectin expression alone may not stratify clinical outcomes.

To assess potential cellular sources of Gal- within the tumor microenvironment, LGALS3 mRNA expression was analyzed across immune cell subsets using ImmucellAI and GSCA with TCGA DLBCL datasets (FIG. 13G). LGALS3 expression correlated positively with macrophage abundance and overall immune infiltration scores (FDR ≤0.05) (FIG. 13H), while exhibiting a negative correlation with B-cell infiltration (FDR ≤0.05) (FIG. 13I). These findings suggested that, in addition to tumor cell-intrinsic expression, Gal-3 may be contributed by macrophage-rich TME.

### Example 5—Gal-3 Binding to Anti-CD19 CAR-T Cells Promoted Cell Death, Whereas Enforced ST6GAL1 Expression in Anti-CD19 CAR-T Cells Diminished Gal-3-Binding and Related Cell Death

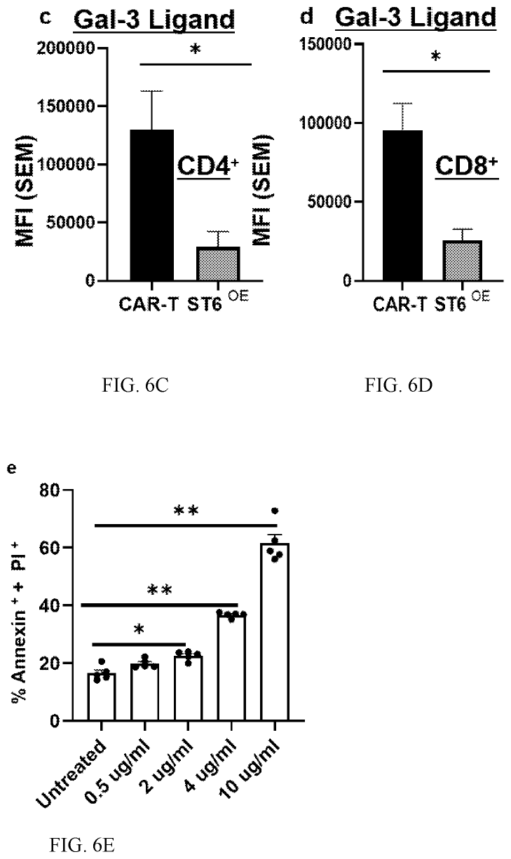

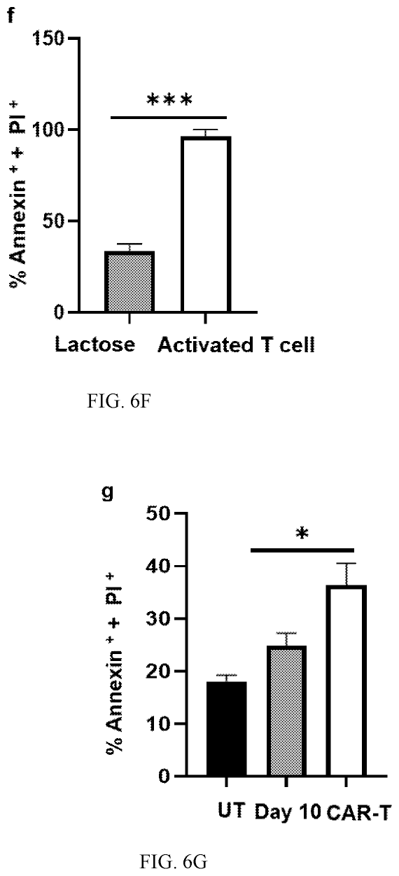

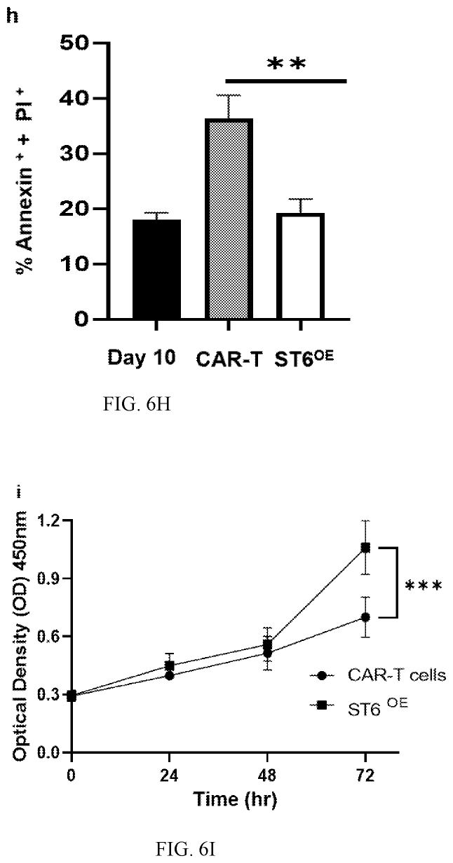

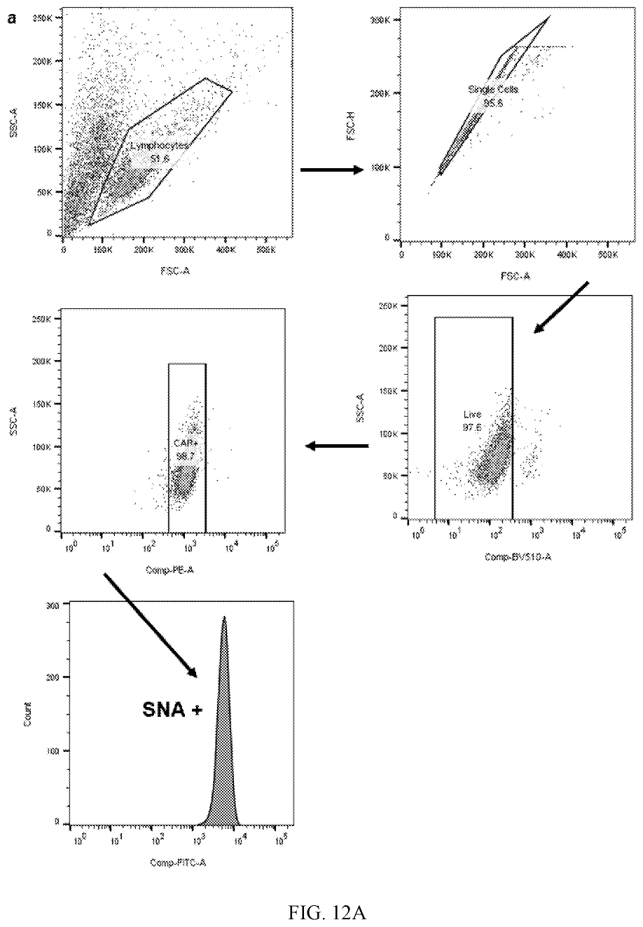

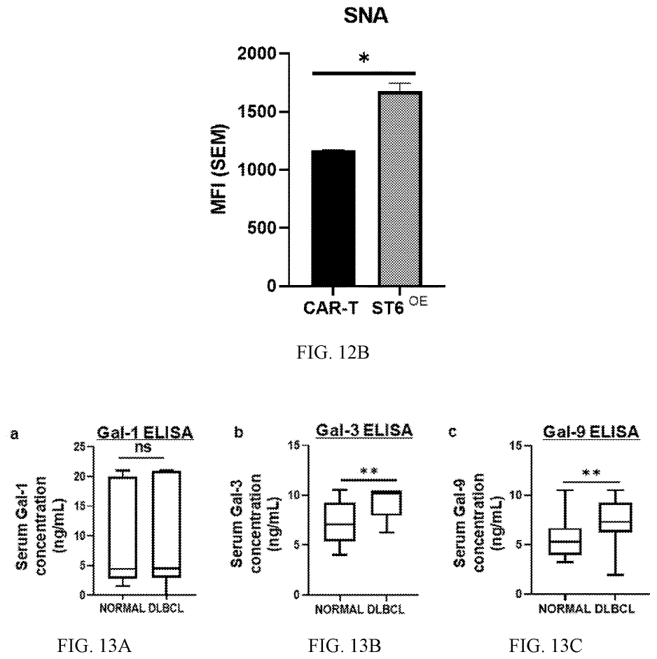

To examine the role of ST6GAL1/α2,6 sialylation expression in modulating Gal-3 binding activity, ST6GAL1/anti-CD19 CAR-T cells (ST6^{OE} CAR-T cells) were generated using a dual ST6GAL1-CD19 CAR (ST6GAL1-P2A-anti-CD19 scFv/4-1BB/CD3ζ) construct. Compared with control CAR-T cells, ST6^{OE} CAR-T cells, which were routinely verified to display increased α2,6 sialylation using SNA-binding (FIGS. 12A and 12B), exhibited significantly reduced rhGal-1 and rhGal-3 binding on both CD4⁺ and CD8⁺ subsets (FIGS. 6A-6D) (p<0.05), indicating that enforced ST6GAL1 expression can modify the glycan landscape of CAR-T cells to reduce Gal-1 and Gal-3 ligand activity. This reduction in Gal-1- and Gal-3-binding suggested that ST6^{OE} CAR-T cells may be less prone to Gal-1- and Gal-3-mediated immunosuppression.

Whether α2,6 sialylation on ST6^{OE} CAR-T cells could inhibit Gal-1- and -3-binding was then determined. Using CD19/BBz or ST6GAL1/CD19/BBz to generate CAR-T cells or ST6^{OE} CAR-T cells as described above, cells with rhGal-1 (10 μg/ml) or rhGal-3 (5 μg/ml) and anti-CD4/8 Abs (n=6 PBMC from donors; OneBlood, Inc) were FACS-analyzed. Both Gal-1 and Gal-3 binding to ST6^{OE} CD4⁺ and CD8⁺ CAR-T cells were significantly blunted compared with T cell controls (p<0.05) (FIGS. 6A-6D), indicating that enforced ST6GAL1 expression can modify the glycan landscape of CAR-T cells to reduce Gal-1 and Gal-3 ligand activity. This reduction in Gal-1- and Gal-3-binding suggested that ST6^{OE} CAR-T cells may be less prone to Gal-1- and Gal-3-mediated immunosuppression.

Given the enrichment of Gal-3 within DLBCL-associated tumor microenvironments, we next investigated whether elevated Gal-3 binding sensitizes anti-CD19 CAR-T cells to Gal-3-mediated cell death. Gal-3 dose-response analyses demonstrated that increasing concentrations of rhGal-3 induced a dose-dependent increase in T-cell apoptosis, as measured by Annexin V and PI (markers of early and late apoptosis, respectively) (FIG. 6E). Consistent with glycan-dependent Gal-3 activity, activated T cells treated overnight with 4 μg/mL rhGal-3 exhibited significantly increased Annexin V⁺/PI⁺ staining compared with lactose-treated controls (FIG. 6F) (p<0.001), in line with prior reports demonstrating extracellular Gal-3-induced T-cell death via glycan binding. Compared with non-transduced Day-10-expanded T cells (CD3/CD28-activated, IL-2-expanded), CD19 CAR-T cells were more sensitive to rhGal-3-induced cell death (FIG. 6G) (p<0.05). To evaluate the role of ST6GAL1 in mitigating Gal-3-mediated apoptosis, Gal-3-induced cell death of ST6^{OE} CAR-T cells vs. control CAR-T cells were compared. The result shows that ST6^{OE} CAR-T cells showed a significant decrease in Annexin V⁺/PI⁺ staining, demonstrating that ST6GAL1 expression conferred protection against Gal-3-induced apoptosis (FIG. 6H) (p<0.01). Furthermore, baseline proliferation by CCK-8 assay showed ST6^{OE} CAR-T cells exhibited significant enhanced proliferative capacity after 72 hr compared with conventional CAR-T cells, indicating improved intrinsic expansion potential (FIG. 6I) (p<0.01).

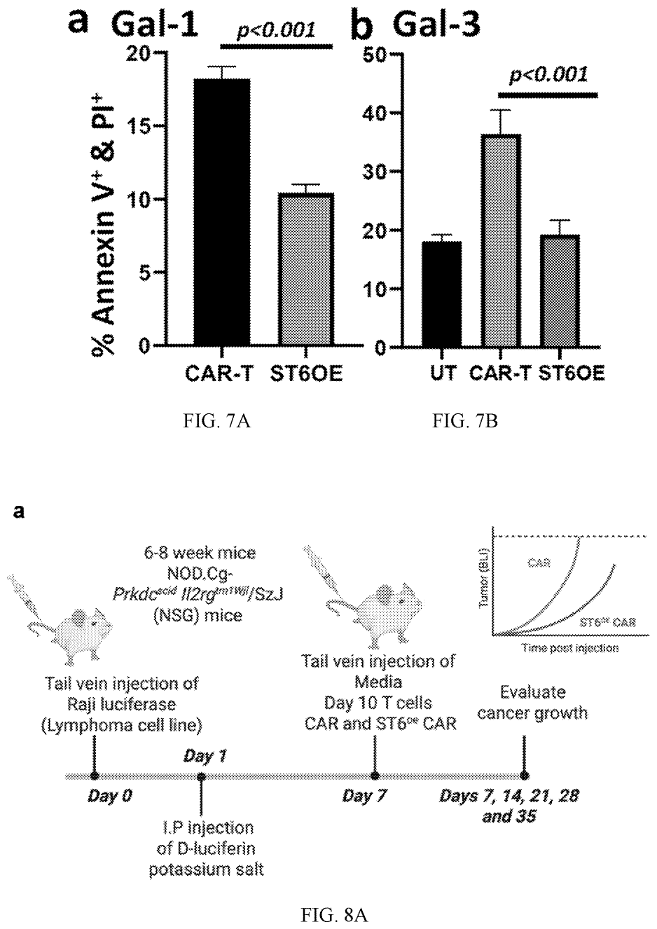

To ascertain whether inhibiting Gal-1 and Gal-3 binding coincides with resistance to Gal-1 and Gal-3-induced cell death, death induction assays were performed with 5 μg/ml rhGal-1 or 4 μg/ml rhGal-3 on CAR-T cells and ST6^{OE} CAR-T cells. After 16 h incubations, cell death was assessed by FACS analysis with Annexin V and PI. Galectins at the cytosolic/apical junction can exist at levels as high as 5 μM or 72.5 μg/ml Gal-1 or 150 μg/ml Gal-3. In the Gal-binding and cell death assays, 4- and 38-fold less levels of rGal-1/-3 respectively were used. In serum and tissues, they typically can be detected in low ng/ml to sub μg/ml range depending on the physio/pathological (i.e. steady-state, inflammation or cancer) condition. Low to sub μg/ml levels commonly found in cancer patient sera were used. Compared with cell death (Annexin V+ and PI+) in Gal-1- and Gal-3-treated CAR-T cells, ST6^{OE} CAR-T cells were relatively resistant to Gal-1- and Gal-3-induced cell death (p<0.001) (FIGS. 7A and 7B). All experiments were performed with 4 different PBMC donors (OneBlood, Inc). In all, these data support the conclusion that α2,6 sialylation can thwart Gal-1- and Gal-3-induced immunomodulation of CAR-T cells.

### Example 6—Gal-3 Induced IL-5 Secretion in Anti-CD19 CAR-T Cells, which was Suppressed by Enforced ST6GAL1 Expression

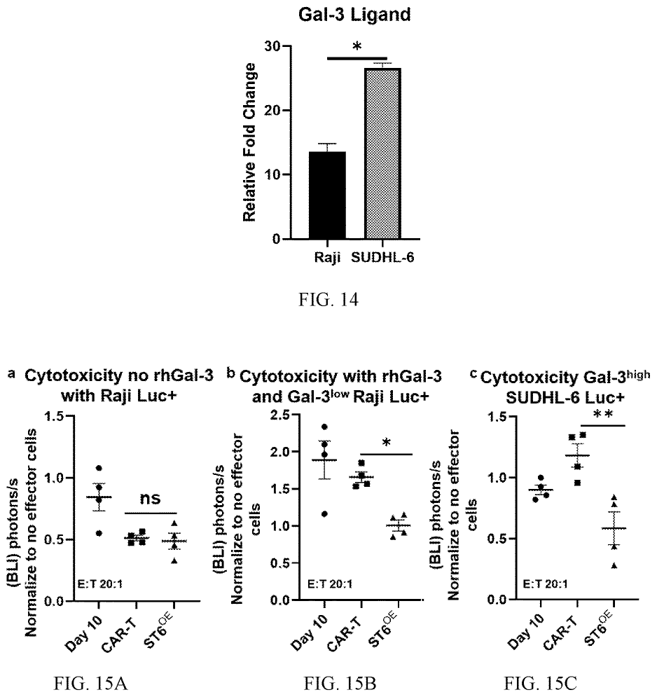

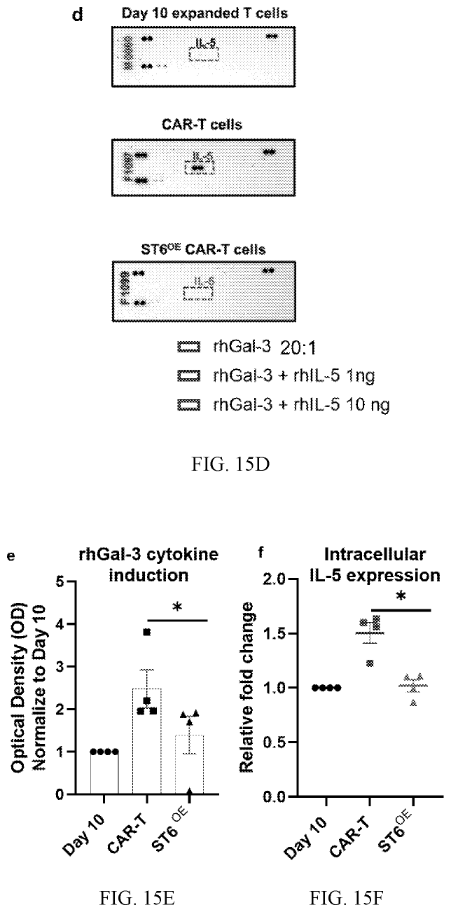

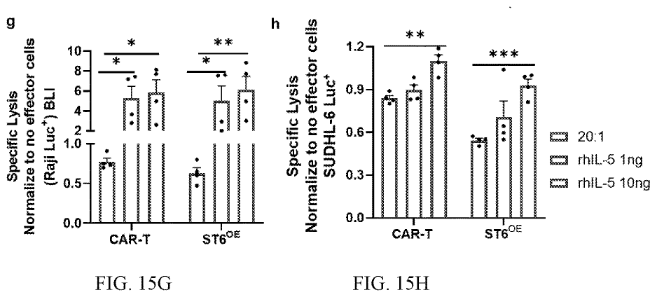

To assess the tumor-killing capacity of ST6^{OE} CAR-T cells vs. control anti-CD19 CAR-T cells, a luciferase-based cytotoxicity assay was performed using CD19⁺ Gal-3^{low} Raji-luciferase (luc)⁺ and Gal-3^{high} SUDHL-6-luc⁺ lymphoma cells (FIG. 14). CAR-T cells and lymphoma cells were co-cultured at effector:target (E:T) ratio of 20:1 for 16 h with or without 4 μg/mL rhGal-3. Post-incubation, CAR-T cells and Raji-luc⁺ co-cultures were lysed using Bright-GLO reagent and luc⁺ tumor viability was measured by bioluminescence imaging (BLI) detection, whose signal was directly proportional to the number of viable luc⁺ target tumor cells and therefore served as an inverse indicator of CAR-T cell-mediated cytotoxicity. In Gal-3-free conditions, both ST6^{OE} CAR-T cells and control CAR-T cells demonstrated comparable levels of Gal-3^{low} Raji-luc⁺ tumor cell killing (FIG. 15A), indicating that enforced ST6GAL1 did not compromise intrinsic cytotoxic function and baseline tumoricidal activity. However, upon Gal-3 exposure to both Gal-3^{low} Raji-luc⁺ cells and Gal-3^{high} SUDHL-6-luc⁺ lymphoma cells, a divergence in cytolytic activity was observed. Compared with control CAR-T cells, ST6^{OE} CAR-T cells exhibited an improved tumoricidal activity, as evidenced by lower BLI signal (p<0.05 and p<0.01, respectively), indicating functional resistance to Gal-3-mediated immunosuppression (FIGS. 15B and 15C). These findings confirmed that enforced ST6GAL1 expression can help protect CAR-T cells from Gal-3-induced immunoregulation, even under conditions of high Gal-3 expression.

Next, the effect of Gal-3 stimulation on anti-CD19 CAR-T cell cytokine responses was investigated. Upon Gal-3 exposure, CAR-T cells exhibited a notable increase in IL-5 secretion, identifying a Gal-3-associated cytokine response in CAR-T cells (FIG. 15D). This IL-5 induction was significantly attenuated in ST6^{OE} CAR-T cells, as confirmed by cytokine profiling (p<0.05) (FIG. 15E) and further validated by intracellular flow cytometry, indicating that enforced ST6GAL1 expression limits Gal-3-driven cytokine skewing (FIG. 15F) (p<0.05). These findings suggest that IL-5 production occurs downstream of Gal-3 engagement and reflects a secondary immunomodulatory consequence of Gal-3 binding rather than a primary initiating mechanism.

Although IL-5 has been associated with eosinophil recruitment and immune modulation in clinical settings, its functional impact on CAR-T cells appears context dependent. To determine whether IL-5 directly contributed to Gal-3-mediated CAR-T cell dysfunction, cytotoxicity assays were performed in the presence of exogenous rhIL-5. Treatment of Raji-luc⁺ cell/SUDHL-6-luc⁺: T cell co-cultures with rhIL-5 and/or rhGal-3 showed that ST6GAL1 overexpression does not confer resistance to IL-5-mediated suppression, as evidenced by increased BLI (p<0.01 and p<0.05, respectively) (FIGS. 15G and 15H). Together, these data demonstrated that ST6GAL1-mediated glycoengineering protects CAR-T cells by limiting upstream Gal-3 engagement and its downstream apoptotic and cytokine-skewing effects but does not preserve CAR-T cell function from IL-5 effects.

### Example 7—ST6^{OE} CAR-T Cell Treatment Exhibited Enhanced Tumor Control, Mouse Survival, and Persistence In Vivo

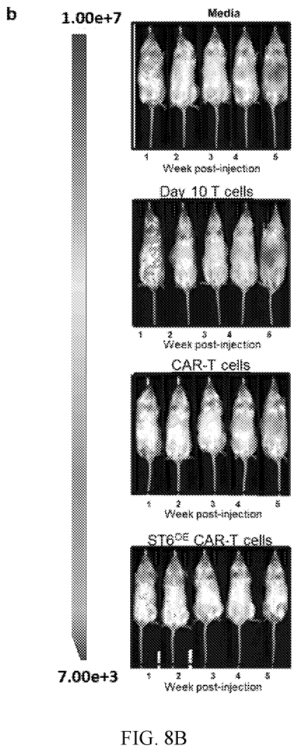

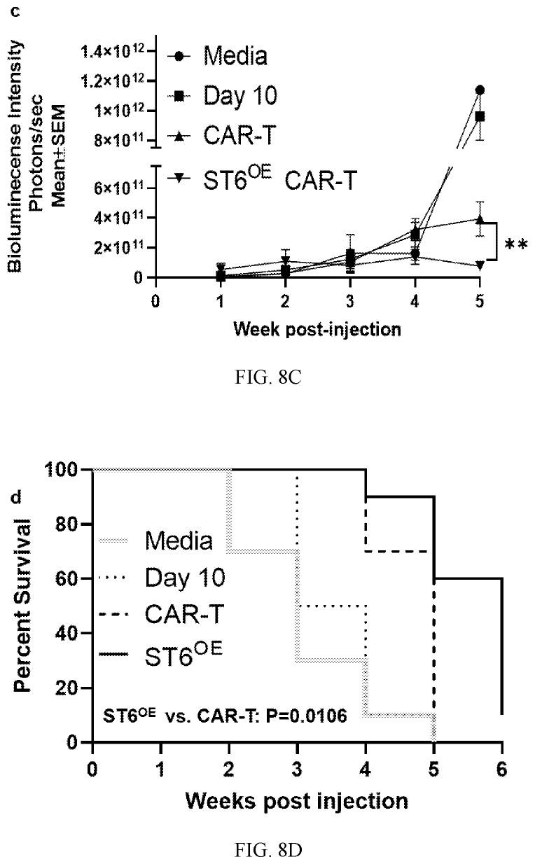

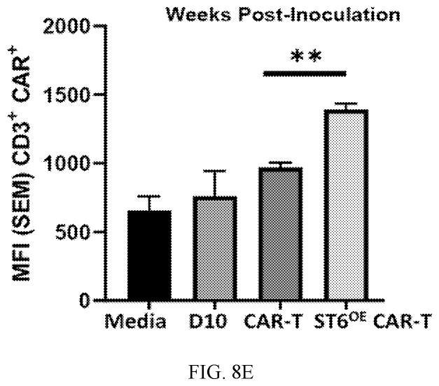

To evaluate in vivo efficacy of ST6^{OE} CAR-T cells, a NOD-SCID IL-2Rγ^{-/-} (NSG) mouse model grafted with CD19⁺ Raji-luc⁺ lymphoma was employed. As depicted in FIG. 8A, mice were injected i.v. with tumor cells on Day 0 and treated i.v. on Day 7 with either media, day 10 T cells, anti-CD19 ST6^{OE} CAR-T cells or control anti-CD19 CAR-T cells. Tumor progression was monitored by BLI detection weekly using AMI HT Imager System until 35 days post-treatment (FIG. 8B). BLI results revealed significantly reduced tumor growth in mice treated with ST6^{OE} CAR-T cells compared to those treated with control CAR-T cells (p<0.01) (FIG. 8C). Kaplan-Meier survival curves showed improved overall survival in the ST6^{OE} CD19 CAR-T cell treatment group compared to control CAR-T cells (p=0.011) (FIG. 8D). CAR-T cell persistence was also assessed by flow cytometric analysis of spleens collected post-mortem. Spleens were harvested, mechanically dissociated, and stained with CAR-specific Abs to identify CAR-T cells. ST6^{OE} CAR T cells were detected at a significantly higher frequency in the spleen compared with control CAR-T cells (p<0.05) (FIG. 8E), suggesting that enforced ST6GAL1 expression enhanced in vivo persistence of CAR-T cells. In all, these results indicated that ST6^{OE} CAR-T cells exhibited improved antitumor activity and persistence in this model.

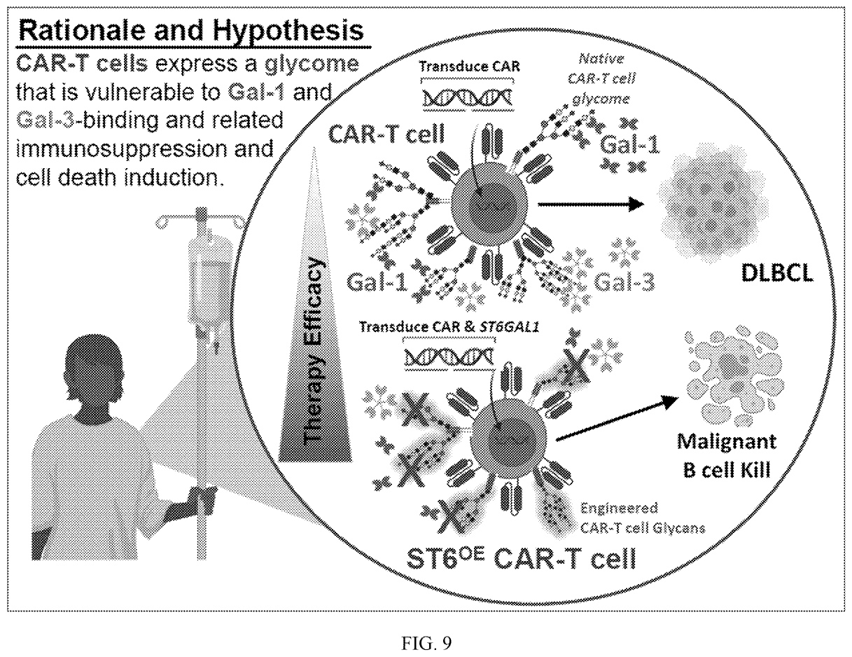

These results support the conclusion that enforced α2,6 sialylation on CAR-T cells can boost in vivo anti-tumor efficacy (FIG. 9).

These data provide strong evidence that Gal-1 and Gal-3 binding to glycans on CAR-T cells is robust, which, unfortunately, compromises cell viability. The CAR-T cell surface glycome and suppressed α2,6 sialylation can provide a therapeutic target to improve CAR-T cell persistence in vivo and efficacy. That is, low α2,6 sialylated N-glycans via low ST6GAL1 action in CAR-T cells promotes Gal-1 and Gal-3-binding and related death induction. Because Gal-1 and Gal-3 binding to its ligands induces CAR-T cell death/dysfunction, interfering with Gal-1 and Gal-3 binding to CAR-T cells by enforcing ST6GAL1 expression increases functional persistence, in vivo (FIG. 10).

The above illustrated examples indicated that CD19 CAR-T cells exhibited extraordinary Gal-1 and Gal-3 binding activity and related Gal-1- and Gal-3-mediated CAR-T cell death and expressed aberrant levels of glycosyltransferases that promote galectin-binding. Conspicuously, expression of α2,6 sialyltransferase 1 (ST6GAL1), which α2,6 sialylates and inhibits Gal-1- and -3-binding glycans, was significantly lower in CAR-T cells compared with control T cell counterparts. These data suggested that CAR-T cells may be specifically susceptible to both Gal-1- and Gal-3-mediated immunomodulation.

To determine whether CAR-T cell glycans avidly bind Gal-1 and -3, which compromise cell viability and function, and whether enforced expression of ST6GAL1 in CAR-T cells can attenuate Gal-1 and -3-binding and improve viability and anti-tumor function, CAR-T cells were engineered to express ST6GAL1 (ST6^{OE} CAR-T) to enforce surface α2,6 sialylation and reduce Gal-1 and Gal-3 binding. In flow cytometry assays, compared with control CAR-T cells, Gal-1 and Gal-3 binding activity and consequent death-inducing properties on ST6^{OE} CAR-T cells was completely blocked (p<0.01). Additionally, using a human DLBCL xenograft mouse model, treatment with ST6^{OE} CAR-T cells was significantly more effective in reducing tumor burden with significantly improved survival time in vivo compared with control CAR-T cell treatments (p<0.001). These findings suggest that enforced ST6GAL1 gene expression can enhance CAR-T cell persistence and anti-tumor efficacy, offering a strategy to improve CAR-T cell therapy outcomes in not only DLBCL patients but in CAR-T cell therapies against other cancers.

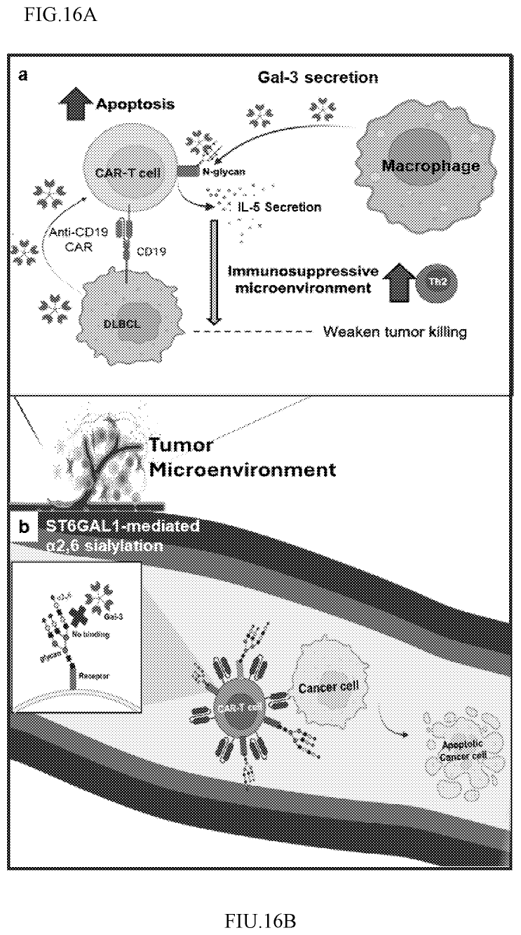

In summary, the data demonstrate that CAR-T cells are intrinsically vulnerable to Gal-3-mediated immunoregulation due to activation-induced remodeling of their glycome and glycan biosynthetic machinery. Enforcing α2,6 sialylation through ST6GAL1 shields CAR-T cells from Gal-3-induced apoptosis and maladaptive cytokine skewing, resulting in enhanced persistence and antitumor efficacy (FIGS. 16A and 16B). This glycoengineering strategy offers an avenue to improve CAR-T cell durability in DLBCL and other malignancies characterized by Gal-1 and/or Gal-3 immunosuppressive microenvironments.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

What is claimed is:

1. An engineered CAR-T cell expressing a chimeric antigen receptor (CAR) and overexpressing an enzyme having glycosyltransferase activity, the CAR comprising an extracellular domain that specifically binds to a cancer antigen, a transmembrane domain, and at least one intracellular co-stimulatory signaling domain.

2. The engineered CAR-T cell of claim 1, the enzyme being α2,6 sialyltransferase (ST6GAL1).

3. The engineered CAR-T cell of claim 1, the cancer antigen being selected from B-cell maturation antigen (BCMA), CD19, CD20, CD22, CD33, CD70, CD123, CD166, mesothelin, CEA, c-Met, PSMA, GD-2, GD-3, FRα, CAIX, CD171, EGFRVIII, HER2, CD133, CEACAM5, CEACAM6, CA-125 (Muc16), sLe^{A} (CA19-9), EGFR, GPC3, PSMA, ROR1, VEGFR2, B7-H3, IL-13Rα, PD-L1, IL-11Rα, EphA2, MAGE, MCAM, NKG2D ligands, TEM1, FAP, GAGE, MUC1, NY-ESO-1, Mesothelin (MSLN), TnMUC1, sialo-Tn (sTn) MUC1, and alpha-folate receptor.

4. The engineered CAR-T cell of claim 1, overexpressing an amino acid sequence of SEQ ID NO: 1 or 2, or an amino acid sequence sharing at least 90% identity with SEQ ID NO: 1 or 2.

5. The engineered CAR-T cell of claim 1, which is ST6^{OE} CAR-T cells comprising ST6GAL1/anti-CD19/BBzeta.

6. A pharmaceutical composition comprising the engineered CAR-T cell of claim 1.

7. A method for treating cancer in a subject, the method comprising administering to the subject having cancer an effective amount of the pharmaceutical composition of claim 6.

8. The method of claim 7, the administration being selected from local, intravenous, intradermal, subcutaneous, and intramuscular routes.

9. The method of claim 7, the composition comprising ST6^{OE} CAR-T cells.

10. The method of claim 7, the subject being a human.

11. The method of claim 7, the engineered CAR-T cells being adoptive anti-tumor T cells derived from the subject's own lymphocytes.

12. The method of claim 9, the ST6^{OE} CAR-T cells being ST6GAL1/anti-CD19/BBzeta T cells.

13. The method of claim 7, the cancer being selected from alveolar rhabdomyosarcoma, bone cancer, brain cancer, breast cancer, chronic myeloid cancer, colon cancer, esophageal cancer, cervical cancer, gastrointestinal cancer, endometrial or hepatocellular carcinoma, hypopharynx cancer, kidney cancer, larynx cancer, liver cancer, lung cancer, malignant mesothelioma, melanoma, multiple myeloma, nasopharynx cancer, ovarian cancer, osteosarcoma, pancreatic cancer, mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer.

14. The method of claim 7, the cancer being hematologic cancer.

15. The method of claim 14, the hematologic cancer being selected from chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma (DLBCL), follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and preleukemia.

16. The method of claim 15, the hematologic cancer being DLBCL.

17. A method for inhibiting galectin-dependent immunosuppression of a CAR-T cell therapy in a subject having cancer, the method comprising administering to the subject having cancer an effective amount of the pharmaceutical composition of claim 6.

18. The method of claim 17, the galectin being galectin-1, galectin-3, and/or galectin-9.

19. The method of claim 17, the cancer being hematologic cancer.

20. The method of claim 19, the hematologic cancer being DLBCL.


SEQUENCE LISTING (2 sequences)

SEQ ID NO: 1 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 175 aa, Homo sapiens
MNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC